(19) United States
(12) Patent Application Publication
(10) Pub. No.: US 2026/0216382 A1
(43) Pub. Date: Jul. 30, 2026

(54) ALPHA-EMITTING RADIONUCLIDES FOR USE IN THE TREATMENT OF PERITONEAL CANCER

(71) Applicant: Oncoinvent AS, Oslo (NO)

(72) Inventor: Roy Hartvig Larsen, Oslo (NO)

(21) Appl. No.: 19/145,466

(22) PCT Filed: Jan. 4, 2024
(86) PCT No.: PCT/EP2024/050149

§ 371 Date: Jul. 2, 2025

(30) Foreign Application Priority Data
Jan. 5, 2023 (EP) 23150472.1
Sep. 28, 2023 (EP) 23200502.5

(51) Int. Cl. A61K 51/04; A61K 47/02; A61P 35/00

(52) U.S. Cl. CPC A61K 51/0478; A61K 47/02; A61P 35/00

(57) ABSTRACT

The present invention relates to pharmaceutical compositions comprising a therapeutically relevant amount of alpha emitting radionuclides, for use in the treatment of cancer, and specifically where the individual has received cytoreductive surgery of one or more peritoneal tumor(s) prior to the administration of the pharmaceutical composition.

### FIELD

The present invention relates to pharmaceutical compositions comprising a therapeutically relevant amount of alpha emitting radionuclides, for use in the treatment of cancer, and specifically where the individual has received cytoreductive surgery of one or more peritoneal tumor(s) prior to the administration of the pharmaceutical composition.

### BACKGROUND

Peritoneal metastases are a significant problem for several cancer types.

Primary cancer and metastatic cancers in the peritoneal cavity are usually treated with chemotherapy, surgery, or radiotherapy or combination of some of these. Although these treatments can give a significant improvement in disease free and overall survival, there is a substantial unmet need for novel therapies to improve the outcome.

Peritoneal cancer can be primary tumours of the peritoneum (peritoneal mesothelioma and primary peritoneal cancer) or disseminate as peritoneal metastasis from tumours of other organs, which include those of intraperitoneal origin (gastric, pancreatic, colorectal, small bowel, ovarian, endometrial, appendiceal cancer as well as sarcoma) and those of extraperitoneal origin (lung, breast and kidney tumours) All of these are candidates for cytoreductive surgery (CRS) with or without hyperthermic intraperitoneal chemotherapy (HIPEC) and alpha-emitter compositions treatment, such as Radspherin.

Therefore, two phase I studies have been conducted with alpha-emitting radioimmunoconjugates to treat patients with peritoneal cancer, although not in combination with surgery or HIPEC. Completed several years ago, these studies did not seem to be followed up with further studies, indicating that the treatment methods were not sufficiently convincing for further clinical development.

Primary colorectal, ovarian, stomach cancer, appendiceal cancer etc, i.e. that is all cancers appearing in tissues that are adjacent to the peritoneal cavity, can be treated relative effectively with cytoreductive surgery (CRS) but disease may reappear later in the form of intraperitoneal and/or distant metastases. Distant metastases may often be effectively treated, by e.g., surgery, external beam radiotherapy (XBR) or chemotherapy. Local intraperitoneal (i.p.) cancer metastases are often difficult to treat effectively. At some institutions the addition of hyperthermic intraperitoneal chemotherapy (HIPEC) following surgery as a two-step procedure that treats certain cancers in the abdomen are used. Cancerous tumors are surgically removed, and then heated chemotherapy drugs are applied directly inside the peritoneal cavity to try to eliminate the remaining cancerous cells. Unfortunately, only relatively limited gains have been seen from adding HIPEC to the surgical procedure. Therefore, there is a significant need for further therapy that can reduce the risk of recurrence of local i.p. metastases.

When cytoreductive surgery is performed on tumors adjacent to the peritoneal cavity a small amount of cells detached from a tumor (e.g. micro seeds) may “leak of” and cause reappearance of local metastases after a few months to a few years. Residual microscopic disease after surgery is also a problem. To counteract this, use of local chemotherapy (e.g. HIPEC) or radiotherapy has been studied but with only minor success. A limiting factor with XBR towards the peritoneal cavity is the high radiosensitivity of some of the adjacent tissues, in particular the intestines, limiting the amount of radiation that can be given. This radiosensitivity is also a limiting factor with longer ranging radiation from radionuclides, e.g. beta-emitters.

It is also a potential problem to irradiate areas with surgical wounds under recovery since the radiation may affect negatively the repair process of the wounds (Gu et al., 1998; Diaz et al., 2021; Haubner et al., 2012). Postoperative radiotherapy is associated with significant adverse effects (Komori et al., 2011). Use of beta-emitting ¹²P Phosphocol colloid particles for local infusion to obtain intraperitoneal irradiation has also been associated with significant complications (Vergote et al., 1993) and after several decades of use was abandoned in most countries due to insufficient effect vs. safety profile (Fields et al., 2017).

It is therefore a need for a new type of radiation therapeutic for local treatment of intraperitoneal cancer cells and micrometastases that can be given at therapeutic effective doses (equivalent doses) without causing harmful side effects such as negative interference with wound healing following surgery.

### SUMMARY

In its broadest aspect, the present invention relates to a pharmaceutical composition comprising a therapeutically relevant amount of alpha emitting radionuclides. The pharmaceutical composition can be for use in the treatment of cancer, and can be wherein the individual has received cytoreductive surgery of one or more peritoneal tumor(s) prior to the administration of the pharmaceutical composition.

In one or more embodiments of the invention, the alpha emitting radionuclides is administered with a radiation dose range of 6 Gy (30 Sv) to 2000 Gy (10 000 Sv), more specifically 10-200 Gy (50-1000 Sv). In one or more embodiments of the invention, the alpha emitting radionuclides is administered with a radiation dose range of 10 Gy (50 Sv) to 200 Gy (1 000 Sv). In one or more embodiments of the invention, the alpha emitting radionuclides is administered with a radiation dose range of 10 Gy (50 Sv) to 1000 Gy (5000 Sv). In one or more embodiments of the invention, the alpha emitting radionuclides is administered with a radiation dose range of 20 Gy (100 Sv) to 500 Gy (2500 Sv). In one or more embodiments of the invention, the alpha emitting radionuclides is administered with a radiation dose range of 10 Gy (50 Sv) to 2000 Gy (10 000 Sv). In one or more embodiments of the invention, the alpha emitting radionuclides is administered with a radiation dose range of 20 Gy (100 Sv) to 200 Gy (1000 Sv). In one or more embodiments of the invention, the alpha emitting radionuclides is administered with a radiation dose range of 10 Gy (50 Sv) to 500 Gy (2500 Sv). In one or more embodiments of the invention, the alpha emitting radionuclides is administered with a radiation dose range of 10 Gy (50 Sv) to 300 Gy (1500 Sv).

In one or more embodiments of the invention, the radionuclide is selected from the group consisting of alpha-radionuclides suitable for therapy consisting of ²²⁴Ra, ²²⁵Ac, ²¹¹At, ²¹³Bi, ²¹²Bi, ²²⁵Ra, ²²³Ra, ¹⁴⁹Tb, ²¹³Pb, ²³⁰U, ²⁵⁵Fm, and ²²⁷Th.

In one or more embodiments of the invention, the radionuclide is selected from the group consisting of alpha-emitting ²²⁴Ra with the progeny radionuclides ²²⁰Rn, ²¹⁶Po, ²¹²Pb, ²¹²Bi, ²¹²Po and ²⁰⁸Tl.

In one or more embodiments of the invention, the radionuclide is a beta emitter with alpha-progenies suitable for therapy, which is ²¹²Pb with progeny radionuclides ²¹²Bi, ²¹²Po and ²⁰⁸Tl.

In one or more embodiments of the invention, the alpha emitting radionuclide is comprised in a particle, which can be biodegradable.

In one or more embodiments of the invention, the particle is comprising a degradable compound and the alpha emitting radionuclide.

In one or more embodiments of the invention, the particles furthermore comprise a phosphorus containing additive.

In one or more embodiments of the invention, the degradable compound is selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent.

In one or more embodiments of the invention, the degradable compound is CaCO₃, such as PEG modified CaCO₃, protein modified CaCO₃ including mAbs and Fabs, carbohydrate modified CaCO₃, lipid modified CaCO₃, vitamin modified CaCO₃, organic compound modified CaCO₃, polymer modified CaCO₃ and/or inorganic crystal modified CaCO₃. In one or more embodiments of the invention, the degradable compound is CaCO₃.

In one or more embodiments of the invention, the phosphorus containing additive is a phosphate selected from the group consisting of orthophosphate, linear oligophosphates and polyphosphates, and cyclic polyphosphates.

In one or more embodiments of the invention, the phosphorus containing additive is a polyphosphate selected from the group consisting of pyrophosphate, tripolyphosphate and triphosphono phosphate.

In one or more embodiments of the invention, the phosphorus containing additive is a cyclic polyphosphate which is sodium hexametaphosphate (SHMP).

In one or more embodiments of the invention, the phosphorus containing additive is a phosphonate.

In one or more embodiments of the invention, the phosphonate is a bisphosphonate.

In one or more embodiments of the invention, the bisphosphonate is selected from the group consisting of Etidronate, Clodronate, Tiludronate, Pamidronate, Neridronate, Olpadronate, Alendronate, Ibandronate, Risedronate, and Zoledronate.

In one or more embodiments of the invention, the phosphonate is a polyphosphonate.

In one or more embodiments of the invention, the polyphosphonate is selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid). In one or more embodiments of the invention, the polyphosphonate is EDTMP-ethylenediamine tetra(methylene phosphonic acid).

In one or more embodiments of the invention, the particle is comprising one or more compounds selected from the group consisting of a polymer, alginate and crystalline salt (including salt of sulfate), silica, phosphate, carbonate, gelatin, polystyrene, lactate, and barium sulfate.

In one or more embodiments of the invention, the size of the particle is from 1 nm to 500 μm.

In one or more embodiments of the invention, the pharmaceutical composition is comprising one or more particles according to the invention and a diluent, carrier, surfactant, and/or excipient.

In one or more embodiments of the invention, the pharmaceutical composition is prepared with an amount of radionuclide that is 1 kBq to 10 GBq per dosing or with an amount of radionuclide that is 50 MBq to 100 GBq suitable for multidose industrial scale production.

In one or more embodiments of the invention, the composition is a particle suspension comprising monodisperse or polydisperse particles as defined herein.

In one or more embodiments of the invention, the cancer is selected from the group consisting of intraperitoneal cancers, intracranial cancers, pleural cancers, bladder cancers, cardiac cancers, cancers in the subarachnoid cavity.

In one or more embodiments of the invention, the cytoreductive surgery of one or more peritoneal tumor(s) is performed pre-administration, e.g., the same day, one day prior to the administration of the pharmaceutical composition comprising a therapeutically relevant amount of alpha emitting radionuclides, such as at least two days, such as at least three days, such as at least four days, such as at least five days, such as at least six days, such as at least seven days, such as at least eight days, such as at least nine days, such as at least ten days, such as at least eleven days, such as at least twelve days, such as at least thirteen days, such as at least fourteen days.

In one or more embodiments of the invention, the pharmaceutical composition is used in combination with other cancer therapies, such as chemotherapy like taxanes (e.g. paclitaxel, docetaxel), platins (e.g. carboplatin, cisplatin), doxorubicin, mitomycin), DNA repair inhibitors such as PARP inhibitors (e.g. Olaparib, Rucaparib, Niraparib, Talazoparib, Veliparib, Pamiparib, CEP 9722, E7016, and 3-Aminobenzamide), and radioimmunotherapies.

In one or more embodiments of the invention, the pharmaceutical composition is a medical device or is comprised in a medical device.

In one or more embodiments of the invention, the concentrations of phosphonates and or phosphate compounds are 1 microgram to 1000 milligram per ml, such as 0.1 mg to 10 mg per ml of final solution, or 1 microgram to 1000 milligram per gram particles in the final solution.

In one or more embodiments of the invention, the individual has received Hyperthermic intraperitoneal chemotherapy (HIPEC).

In one or more embodiments of the invention, the pharmaceutical composition is administered to an individual in need thereof.

### DETAILED DESCRIPTION

In the current work, the present inventor has shown that alpha-emitting radionuclide(s) have the unique property of being able to treat cancer in individuals that have received cytoreductive surgery of one or more peritoneal tumor(s) prior to the administration of the alpha-emitting radionuclide(s).

The alpha-emitting radionuclide(s) can be incorporated in particles. The main purpose of this modality is the high local retention the particles carrying the alpha-emitting source, minimizing risk of systemic toxicity, and the short range of the alpha particles which may limit the radiation to deeper regions of tissues adjacent to the peritoneum, potentially reducing the risk of intestinal toxicity. Preclinical testing of various versions of ²²⁴Ra-labeled microparticles was recently presented (Li et al., 2021). However, some factors, including the clinically relevant amounts of particles and the number of radionuclides per particle in relation to disease progression, toxicity and dosimetry in humans would have to be determined more exactly based on clinical data.

One significant unknown factor, that was not tested in preclinical models, was the use of radiation in patients with newly created surgical wounds from removal of cancer tissues. It is known in the field that radiation of body areas with wounds can be problematic as the irradiation may cause inhibition of recovery etc. It could be expected that the very focused nature of short-ranging alpha-particles irradiating the surfaces of newly operated peritoneal areas could be particularly problematic due to the potential detrimental effects off alpha-particle irradiation on wound healing (Xueting et al., 2019). It was therefore astonishing to learn when the present inventor herein did the dosimetric assessment that very high radiation dose equivalents could be given in this setting without causing increased frequency of serious adverse events.

It was a surprising finding that when dosimetry calculations was performed on clinically tested activity levels found to be safe, very high doses, in terms of dose equivalents (Sieverts; Sv) of alpha particle radiation, could be given i.p. without significant toxicity to patients. It is also shown herein by clinical data that it is possible to strongly limit the recurrence of local i.p. metastases when alpha emitter is administered following surgical resection of tumors from cancers in tissues adjacent to the i.p. cavity as demonstrated by data from colorectal cancer and ovarian cancer patients.

Thus, the present invention relates to a pharmaceutical composition comprising a therapeutically relevant amount of alpha emitting radionuclides. The pharmaceutical composition can be used in the treatment of cancer, and can be wherein the individual has received cytoreductive surgery of one or more peritoneal tumor(s) prior to the administration of the pharmaceutical composition. In one or more embodiments of the invention, the individual has received Hyperthermic intraperitoneal chemotherapy (HIPEC).

It is quite common for suspensions of microparticles with some sedimentation and flocculation etc causing a macroscopic “grain” of particles which can potential cause a radiation “hot spot”. The present inventor has shown herein that very little alpha radiation escapes this for the therapeutic relevant configuration of alpha emitter, e.g. Radspherin, e.g., 10 MBq ²²⁴Ra per gram of calcium carbonate (i.e., exemplified as approximately 126 ²²⁴Ra atoms per 3 μm microparticle, see examples), thus contributing to the favourable safety profile of the product.

Too many radioactive atoms per particle, exemplified as such as 20 000 or 100 000 atoms per 3 μm particle significantly increase the risk of “hot spot” toxicity complications. Too few atoms per particle, such as 1 atom or lower par particle, cause a too large degree of self-absorption of the radiation, since a very high amount of particles would be required to cover the peritoneal areas and liquid volumes with sufficient level of radioactivity. It is therefore advantageous and clinically relevant to optimize the particle elements, and ensure that there are more than 1 atom on average per 3 μm particle, such as more than 5 atoms on average per 3 μm particle, such as more than 10 atoms on average per 3 μm particle, such as more than 25 atoms on average per 3 μm particle, such as more that 50 atoms on average per 3 μm particle, and less than 100.000 atoms on average per 3 μm particle, such as on average 20.000 atoms per 3 μm particle, such as on average 10.000 atoms per 3 μm particle, such as on average 10.000 atoms per 3 μm particle, such as on average 50 atoms per 3 μm particle. An aspect of the present invention relates to a particle as described herein where there is/are more than 1 atom on average per 3 μm particle. In one or more embodiments of the present invention, there are more than 5 atoms on average per 3 μm particle. In one or more embodiments of the present invention, there are more than 10 atoms on average per 3 μm particle. In one or more embodiments of the present invention, there are more than 25 atoms on average per 3 μm particle. In one or more embodiments of the present invention, there are more that 50 atoms on average per 3 μm particle, and less than 100.000 atoms on average per 3 μm particle. In one or more embodiments of the present invention, there are 20.000 atoms on average per 3 μm particle. In one or more embodiments of the present invention, there are 10.000 atoms on average per 3 μm particle. In one or more embodiments of the present invention, there are 10.000 atoms on average per 3 μm particle. In one or more embodiments of the present invention, there are 50 atoms on average per 3 μm particle. This particle can be clinically relevant in a pharmaceutical composition, and for the uses described herein.

In one or more embodiments of the invention, the pharmaceutical composition is administered to an individual in need thereof.

The present invention relates to the an alpha emitter comprises in a particle, such as in a particle used in suspension. The particle can be a microparticle. The particles can be of an appropriate size, with a number of radioactive atoms per particle, and total amount in relation to surface area to be treated. The alpha emitting compound, such as in a microparticle suspension, is appropriate for human therapeutic use with an acceptable toxicity profile even in newly operated patients with surgical wounds in the early process of healing.

The examples show that ²²⁴Ra-labeled microparticles could deliver unprecedented level of radiation equivalent doses without harmful side effects and with an observable suppression of cancer recurrences in the peritoneal cavity. The necessary parameters for clinical usefulness is described, including size of particles, numbers of radioactive atoms required per microparticle, the amount of particles in gram necessary to cover the surface area of the peritoneum for sufficient dose distribution etc. By fine tuning these parameters for clinical use, a pharmaceutical composition comprising an alpha emitter, such as a ²²⁴Ra-labeled microparticles in suspension, showing relevant antitumor activity and acceptable toxicity in patients were discovered.

The alpha emitting radionuclides of the present invention, such as in microparticles, can be administered by use of a catheter ensuring intracavitary administration. The size of the particles would substantially cause a local retention to diminish systemic exposure. Such as 10-100% retention, such as 50-100% retention, such as 60-80% retention of the radionuclide.

The particle of the present invention may be autoclavable. The particle can be used for preparing suspensions for clinical use in radiotherapy in cancer patients with peritoneal cancer following cytoreductive surgery to remove macroscopic tumors. The inventor found that the alpha-emitter, such as ²²⁴Ra-labeled particles in suspension, can be used for treatment against peritoneal micrometastases and tumor fragments and cells leaked during surgery.

The pharmaceutical composition of the present invention, such as Radspherin, can be used in a catheter administration procedure for intracavitary administration. The administration can be i.p. administration. Local administration as described can be advantageous over intravenous administration.

In the examples, it was found in the current work that 0.7 grams of microparticles with size of about 3 um (numerical) diameter containing about 126 radioactive atoms of ²²⁴Ra per particle, were quite optimal in terms of therapeutic activity and normal tissue toxicity when administered intraperitoneally to patients following cytoreductive surgery (CRS). This treatment could also be used after CRS in combination with HIPEC without observable increase in adverse events, indicating that, alpha emitting radionuclides, exemplified by Radspherin, was very suitable as add on treatment to inactivate micrometastases and leaked cells in body cavities following standard therapies removing the macroscopic tumors. In one or more embodiments of the present invention, there are 0.1 gram to 10 gram of microparticles in each therapeutically relevant pharmaceutical dose. In one or more embodiments of the present invention, there are 0.5 gram to 5 gram of microparticles in each therapeutically relevant pharmaceutical dose. In one or more embodiments of the present invention, there are 0.5 gram to 10 gram of microparticles in each therapeutically relevant pharmaceutical dose. In one or more embodiments of the present invention, there are 0.1 gram to 5 gram of microparticles in each therapeutically relevant pharmaceutical dose.

The radionuclide in the particles of the present invention can therefore be tailored according to the intended use.

The main medical advantages of alpha particle emitting compounds in local therapy in e.g., the intraperitoneal cavity is the shorter range, typically less than 0.1 mm for alphas compared with mm to cm ranges for beta-particles from medical beta emitters.

Use of alpha-emitters would in an intracavitary setting reduce risk for toxicity due to irradiation of deeper regions of internal organs like the radiosensitive intestinal crypt cells in the case of intraperitoneal (IP, i.p.) use. Also is the high linear energy transfer of the emitted alpha particles advantageous since very few alpha hits are needed to kill a cell and cellular resistance mechanism like high repair capacity for DNA strand breaks is less of a problem because of the high probability of producing irreparable double strand breaks.

The high effect per decay means less radioactivity is needed reducing the need for shielding of hospital staff and relatives since most alpha- and beta emitters also emit some X-rays and gammas which needs to be shielded against.

In situations where the cancer is characterized as a bulky disease, the longer range of beta particles compared to alpha particles may be advantageous. The longer path length of beta-particles can result in the so-called cross-fire effect, where irradiation of a significantly higher portion of neighboring and distant cells, causing damage to cells that are further away from to the radiolabeled particle occurs.

In the present context, progeny is understood as the radionuclides that are the result of the decay of a parent radionuclide. Thus, when for example ²²⁴Ra is the parent radionuclide, ²²⁰Rn (the daughter radionuclide), ²¹⁶Po (the granddaughter radionuclide), and ²¹²Pb (the great granddaughter radionuclide). ²²⁰Rn, ²¹⁶Po and ²¹²Pb are therefore all considered progeny radionuclides of ²²⁴Ra.

Thus, in one embodiment is the alpha-emitting radionuclide ²²⁴Ra with the daughter radionuclide ²²⁰Rn, the granddaughter radionuclide ²¹⁶Po, and the great granddaughter radionuclide ²¹²Pb. For the particle of the present invention will these all be comprised by the particle when ²²⁴Ra is the radionuclide.

In one or more embodiments of the invention, the radionuclide is selected from the group consisting of alpha-radionuclides suitable for therapy consisting of ²²⁴Ra, ²²⁵Ac, ²¹¹At, ²¹³Bi, ²¹²Bi, ²²⁵Ra, ²²³Ra, ¹⁴⁹Tb ²¹³Pb ²³⁰U, ²⁵⁵Fm, and ²²⁷Th. In one or more embodiments of the invention, the radionuclide is ²²⁴Ra. In one or more embodiments of the invention, the radionuclide is ²²⁵Ac.

In one or more embodiments of the invention, the radionuclide is ²¹¹At. In one or more embodiments of the invention, the radionuclide is ²¹³Bi. In one or more embodiments of the invention, the radionuclide is ²¹²Bi. In one or more embodiments of the invention, the radionuclide is ²²⁵Ra. In one or more embodiments of the invention, the radionuclide is ²²³Ra. In one or more embodiments of the invention, the radionuclide is ¹⁴⁹Tb. In one or more embodiments of the invention, the radionuclide is ²¹³Bi. In one or more embodiments of the invention, the radionuclide is ²¹³Bi. In one or more embodiments of the invention, the radionuclide is ²²⁵Ra. In one or more embodiments of the invention, the radionuclide is ²³⁰U. In one or more embodiments of the invention, the radionuclide is ²⁵⁵Fm. In one or more embodiments of the invention, the radionuclide is ²²⁷Th.

In one or more embodiments of the invention, the radionuclide is selected from the group consisting of alpha-emitting ²²⁴Ra with the progeny radionuclides ²²⁰Rn, ²¹⁶Po, ²¹²Pb, ²¹²Bi, ²¹²Po and ²⁰⁸Tl.

In one or more embodiments of the invention, the radionuclide is a beta emitter with alpha-progenies suitable for therapy, which is ²¹²Pb with progeny radionuclides ²¹²Bi, ²¹²Po and ²⁰⁸Tl.

In one or more embodiments of the invention, the alpha emitting radionuclide is comprised in a particle, which can be biodegradable. In one or more embodiments of the invention, the particle is comprising a degradable compound and the alpha emitting radionuclide. In one or more embodiments of the invention, the particles furthermore comprise a phosphorus containing additive.

Thus, the present invention relates to a particle comprising a degradable compound, a radionuclide, and a phosphorus containing additive. The phosphorus containing additive can be incorporated into the particle, be associated with the surface of the particle or be present in the surroundings of the particle, i.e. in the composition or suspension that the particle is part of. Thus, one aspect of the present invention relates to a composition or suspension comprising a particle, wherein the particle comprises a degradable compound, a radionuclide and a phosphorus containing additive, and wherein the phosphorus containing additive is associated with the particle by being present in the composition or suspension. The presence can be as part of the particle. The presence can be on the surface of the particle. The presence can be in the dispersion of the particle. The presence can be as part of the composition or suspension and/or dispersion of particles. The presence can also be as part of the particle and as part of the composition or suspension of particles. These individual components can be combined into different types of particles that have different characteristics depending on the intended use of the particles.

This means that the phosphorus containing additive, such as EDTMP and/or pamidronate, normally will be found at least in trace amounts on or associated with the individual particles. The phosphorus containing additive, such as EDTMP and/or pamidronate, will therefore normally be at least partly found on or in the particle if particles in a composition, such as a solution, are tested for content of the phosphorus containing additive. The total amount of phosphorus containing additive, such as EDTMP and/or pamidronate, in a composition (such as a solution) will vary depending on the particle design, but will usually mean that at least 0.01-80% of the total amount of phosphorus containing additive in the composition will be in or on the particles, and the remaining part will be in the composition. The at least 0.01-80% of the total amount of phosphorus containing additive in the composition that will be in or on the particles can be 0.1-50%, such as 10-50%, such as 20-80%, such as 10-80%.

The degradable compound of the present invention can be any compound that can be degraded. The degradation can be done by any route selected from the group consisting of high pH, low pH, temperature, proteases, enzymes, nucleases and/or by cellular processes like endocytosis, which also includes phagocytosis. The degradable compounds can therefore be non-toxic salt or a crystal of a non-toxic salt.

In one or more embodiments of the invention, the particle is comprising one or more compounds selected from the group consisting of a polymer, alginate and crystalline salt (including salt of sulfate), silica, phosphate, carbonate, proteins including albumin and gelatin, polystyrene, lactate, and barium sulfate. In one or more embodiments of the invention, the particle is comprising a polymer. In one or more embodiments of the invention, the particle is comprising alginate. In one or more embodiments of the invention, the particle is comprising crystalline salt (including salt of sulfate). In one or more embodiments of the invention, the particle is comprising silica. In one or more embodiments of the invention, the particle is comprising gelatin. In one or more embodiments of the invention, the particle is comprising polystyrene. In one or more embodiments of the invention, the particle is comprising lactate.

In one or more embodiments of the invention, the degradable compound is selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent.

Major constituent is defined as at least 20% of the total molecular weight of the particle, such as at least 30% of the total molecular weight of the particle, such as at least 40% of the total molecular weight of the particle, such as at least 50% of the total molecular weight of the particle, such as at least 60% of the total molecular weight of the particle, such as at least 70% of the total molecular weight of the particle, such as at least 80% of the total molecular weight of the particle, such as at least 90% of the total molecular weight of the particle, such as at least 95% of the total molecular weight of the particle, such as at least 98% of the total molecular weight of the particle, such as at least 99% of the total molecular weight of the particle.

In one or more embodiments of the invention, the degradable compound is CaCO₃, such as PEG modified CaCO₃, protein modified CaCO₃ including mAbs and Fabs, carbohydrate modified CaCO₃, lipid modified CaCO₃, vitamin modified CaCO₃, organic compound modified CaCO₃, polymer modified CaCO₃ and/or inorganic crystal modified CaCO₃.

The degradable compound can be MgCO₃ which is selected from the group consisting of PEG modified MgCO₃, protein modified MgCO₃ including mAbs and Fabs, carbohydrate modified MgCO₃, lipid modified MgCO₃, vitamin modified MgCO₃, organic compound modified MgCO₃, polymer modified MgCO₃ and/or inorganic crystal modified MgCO₃.

The degradable compound can be SrCO₃ which is selected from the group consisting of PEG modified SrCO₃, protein modified SrCO₃ including mAbs and Fabs, carbohydrate modified SrCO₃, lipid modified SrCO₃, vitamin modified SrCO₃, organic compound modified SrCO₃, polymer modified SrCO₃ and/or inorganic crystal modified SrCO₃.

The degradable compound can be BaCO₃ which is selected from the group consisting of PEG modified BaCO₃, protein modified BaCO₃ including mAbs and Fabs, carbohydrate modified BaCO₃, lipid modified BaCO₃, vitamin modified BaCO₃, organic compound modified BaCO₃, polymer modified BaCO₃ and/or inorganic crystal modified BaCO₃.

The degradable compound can be Ca₅(PO₄)₃(OH) which is selected from the group consisting of PEG modified Ca₅(PO₄)₃(OH), protein modified Ca₅(PO₄)₃(OH) including mAbs and Fabs, carbohydrate modified Ca₅(PO₄)₃(OH), lipid modified Ca₅(PO₄)₃(OH), vitamin modified Ca₅(PO₄)₃(OH), organic compound modified Ca₅(PO₄)₃(OH), polymer modified Ca₅(PO₄)₃(OH) and/or inorganic crystal modified Ca₅(PO₄)₃(OH).

The degradable compound can be fluoroapatite which is selected from the group consisting of PEG modified fluoroapatite, protein modified fluoroapatite including mAbs and Fabs, carbohydrate modified fluoroapatite, lipid modified fluoroapatite, vitamin modified fluoroapatite, organic compound modified fluoroapatite, polymer modified fluoroapatite and/or inorganic crystal modified fluoroapatite.

The composite particles can comprise two or more of these degradable compounds where they combined are a major constituent, as defined above.

The degradable compounds may be used as composites with other salts or proteins or peptides and subject to surface modification by surfactants like oleates and similar.

In a special embodiment, the degradable compounds are used with a compound selected from the group consisting of poly ethylene glycol (PEG) modified particles of the degradable compound or inorganic crystal modified degradable compound.

In a special embodiment the degradable compounds are modified with functional receptor and or antigen binding groups, including monoclonal antibodies and derivatives and vitamins and derivatives allowing receptor or antigen binding of particle to individual target cells and diseased tissues. This means that modifications of the particles relate to the addition of other compounds to degradable compounds. This can be done in various ways, and through interactions such as dipole-dipole interactions, ion-dipole and ion-induced dipole forces, hydrogen bonding, Van der Waals forces, and relative strength of forces.

A chelator can be used, preferentially conjugated to a target affinic molecule, e.g., monoclonal or polyclonal antibody or derivatives of antibody, vitamins or derivatives of vitamins.

Monoclonal antibodies (mAbs), polyclonal antibodies (pAbs), antigen-binding fragments (Fabs) and other types of polypeptides and proteins can be used to include specific targeting in the particle, i.e. by adding a specific targeting molecule, the particles will be able to have enhanced affinity for certain target cells in the body.

The phosphorus containing additive can be a phosphate, thus becoming a phosphate containing additive. The phosphorus containing additive can also be a phosphonate, thus becoming a phosphonate containing additive. In one or more embodiments of the invention, the phosphorus containing additive is a phosphate selected from the group consisting of orthophosphate, linear oligophosphates and polyphosphates, and cyclic polyphosphates.

In one or more embodiments of the invention, the phosphorus containing additive is a polyphosphate selected from the group consisting of pyrophosphate, tripolyphosphate and triphosphono phosphate.

Phosphonates and phosphonic acids are organophosphorus compounds containing C—PO(OH)₂ or C—PO(OR)₂ groups (where R=alkyl, aryl). Phosphonic acids, typically handled as salts, are generally non-volatile solids that are poorly soluble in organic solvents, but soluble in water and common alcohols. Thus, the various salts and acids of the phosphonates are also considered parts of the definition of phosphonate.

A phosphoric acid, in the general sense, is a phosphorus oxoacid in which each phosphorus atom is in the oxidation state +5, and is bonded to four oxygen atoms, one of them through a double bond, arranged as the corners of a tetrahedron. Removal of the hydrogen atoms as protons H⁺ turns a phosphoric acid into a phosphate anion. Partial removal yields various hydrogen phosphate anions.

The phosphorus containing additive can be a phosphonate. The phosphonate can be a bisphosphonate. The bisphosphonate can be selected from the group consisting of Etidronate, Clodronate, Tiludronate, Pamidronate, Neridronate, Olpadronate, Alendronate, Ibandronate, Risedronate, and Zoledronate. In one or more embodiments of the present invention the bisphosphonate is Etidronate. In one or more embodiments of the present invention the bisphosphonate is Clodronate. In one or more embodiments of the present invention the bisphosphonate is Tiludronate. In one or more embodiments of the present invention the bisphosphonate is Pamidronate. In one or more embodiments of the present invention the bisphosphonate is Neridronate. In one or more embodiments of the present invention the bisphosphonate is Olpadronate. In one or more embodiments of the present invention the bisphosphonate is Alendronate. In one or more embodiments of the present invention the bisphosphonate is Ibandronate. In one or more embodiments of the present invention the bisphosphonate is Risedronate. In one or more embodiments of the present invention the bisphosphonate is Zoledronate.

The phosphonate can be a polyphosphonate. The polyphosphonate can be selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid). In one or more embodiments of the present invention the phosphonate is EDTMP-ethylenediamine tetra(methylene phosphonic acid). In one or more embodiments of the present invention the phosphonate is DOTMP-1,4,7,10-Tetraazacyclododecane-1,4,7,10-tetrayl-tetrakis(methylphosphonic acid). In one or more embodiments of the present invention the phosphonate is DTPMP-diethylenetriaminepenta(methylene-phosphonic acid). Thus, the phosphonate can be EDTMP-ethylenediamine tetra(methylene phosphonic acid).

In one embodiment, the particle of the present invention comprises CaCO₃ as the degradable compound, EDTMP-ethylenediamine tetra(methylene phosphonic acid) as the phosphorus containing additive, and ²²⁴Ra as the radionuclide.

In one embodiment, the particle of the present invention comprises CaCO₃ as the degradable compound, Pamidronate as the phosphorus containing additive, and ²¹²Pb as the radionuclide.

The phosphate containing additives can be selected from the group consisting of orthophosphate, linear oligophosphates and polyphosphates, and cyclic polyphosphates. The polyphosphate can be selected from the group consisting of pyrophosphate, tripolyphosphate and triphosphono phosphate. The phosphorus containing additive can be a cyclic polyphosphate which for example can be sodium hexametaphosphate (SHMP).

In one or more embodiments of the invention, the phosphorus containing additive is a cyclic polyphosphate which is sodium hexametaphosphate (SHMP). In one or more embodiments of the invention, the phosphorus containing additive is a phosphonate. In one or more embodiments of the invention, the phosphonate is a bisphosphonate. In one or more embodiments of the invention, the bisphosphonate is selected from the group consisting of Etidronate, Clodronate, Tiludronate, Pamidronate, Neridronate, Olpadronate, Alendronate, Ibandronate, Risedronate, and Zoledronate. In one or more embodiments of the invention, the phosphonate is a polyphosphonate.

In one or more embodiments of the invention, the polyphosphonate is selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid).

The concentrations of phosphonates and or phosphate compounds are 1 microgram to 1000 milligram per ml, such as 0.1 mg to 10 mg per ml of final solution, or 1 microgram to 1000 milligram per gram particles in the final solution.

Thus, for phosphate containing additives, such as EDTMP, the range for a patient dose can be 1 ug to 1 g per mL or 1 ug to 1 g per g particle, for instance a patient dose could be of 2.5 mg/mL or 25 mg/gram degradable compound, such as CaCO₃. The addition of phosphate containing additives, such as EDTMP, can be 0.1-10%. The addition can also be 0.5-5% or 0.5-2%. The addition can be at least 1%.

The phosphate containing additive, such as EDTMP, can be used in a concentration of 24.4 mg phosphate containing additive, such as EDTMP, for a 10 ml product, with for instance a range of 20 to 30 mg per 10 mL product.

The phosphate containing additive, such as EDTMP, can be used as and an excipient with a final concentration of 5.6 mM (2.44 mg/mL), with a range of 4.6-6.6 mM.

The phosphate containing additive, such as EDTMP, can be used at 24.4 mg/g calcium carbonate, or a range of 10 mg-50 mg per gram calcium carbonate

The phosphate containing additive, such as EDTMP, can be used at 2.4% w/w, with a range of 1-20% w/w.

The amount of phosphate containing additive, such as EDTMP, can be 24.4 mg in 10 ml product.

The phosphate containing additive, such as EDTMP or EDTMPA (acid), can be used as an excipient with a concentration of 5.6 mM (2.44 mg/mL).

The concentration of the phosphate containing additive, such as EDTMP or EDTMPA (acid), can be 5.6 mM±15%.

For phosphonate containing additives, such as Pamidronate, the range for a patient dose can be 1 ug to 1 g per mL or 1 ug to 1 g per g particle, for instance a patient dose could be of 2.5 mg/mL or 25 mg/gram degradable compound, such as CaCO₃. The addition of phosphate containing additives, such as Pamidronate, can be 0.1-10%. The addition can also be 0.5-5% or 0.5-2%. The addition can be at least 1%.

The phosphate containing additive, such as Pamidronate, can be used in a concentration of 10 mg phosphate containing additive, such as Pamidronate, for a 10 ml product, with for instance a range of 5 to 50 mg per 10 mL product.

The phosphate containing additive, such as Pamidronate, can be used as and an excipient with a final concentration of 4 mM (1 mg/mL), with a range of 0.1-10 mM.

The phosphate containing additive, such as Pamidronate, can be used at 0.01 g/g calcium carbonate, or a range of 1 mg-50 mg per gram calcium carbonate

The phosphate containing additive, such as Pamidronate, can be used at 1% w/w, with a range of 0.1-5% w/w.

The amount of phosphate containing additive, such as Pamidronate, can be 10 mg in 10 ml product.

The phosphate containing additive, such as Pamidronate or Pamidronic acid, can be used as an excipient with a concentration of 4 mM (1 mg/mL).

The concentration of the phosphate containing additive, such as Pamidronate or Pamidronate acid, can be 4 mM±25%.

These radionuclides can be combined in the particles of the present invention, so the particle comprises one, two or more of the above-mentioned radionuclides. This can happen by natural causes where a radionuclide decays and therefore becomes its natural progeny. Such situation can for example happen when ²²⁴Ra is the parent radionuclide, ²²⁰Rn (the daughter radionuclide), ²¹⁶Po (the granddaughter radionuclide), and ²¹²Pb (the great granddaughter radionuclide). ²²⁰Rn, ²¹⁶Po and ²¹²Pb are therefore all considered progeny radionuclides of ²²⁴Ra and will due to the natural decay of ²²⁴Ra automatically be present in the particles in certain amounts.

Two or more radionuclides can also be comprised in the particle because it can be beneficial to have higher amounts than from the natural decay for the intended use of the particle. This can for example happen if ²²⁴Ra and ²¹²Pb are mixed for the formation of the particles. There will in this situation be a higher level of ²¹²Pb in the particle than there would be if the particle was prepared with purified ²²⁴Ra.

The amount of radionuclide used per patient dosage may be in the range of 1 kBq to 10 GBq more preferably 100 kBq to 100 MBq, event more preferably range is 0.5 MBq to 25 MBq. Range dosage can be 10 MBq to 10 GBq per patient dose. Range dosage can be 10 MBq to 5 GBq per patient dose. The ranges can be for beta emitters, alpha emitters or combinations hereof. The ranges can be for therapy or imaging. Dosage will depend on the cancer type, and for example how aggressive the disease is. In one embodiment is the dosage 10-100 kBq/kg, such as 20-50 kBq/kg. In another embodiment is the dosage 10-1000 kBq/kg, such as 25-300 kBq/kg. In a further embodiment the is the dosage 100-500 kBq/kg, such as 150-300 kBq/kg. In one embodiment the dosage is 1-100 MBq/kg, such as 5-20 MBq/kg. In another embodiment the dosage is 1-1000 MBq/kg, such as 10-50 MBq/kg. In a further embodiment the is the dosage 100-500 MBq/kg, such as 150-300 MBq/kg.

In one embodiment of the present invention is the pharmaceutical composition prepared with an amount of radionuclide that is 1 kBq to 10 GBq per dosing. For instance, if 100 patient doses are produced in one batch per day this could be made up of a total of 1-10 GBq divided into 100 single dosing vials or ready to use syringes.

In another embodiment of the present invention is the pharmaceutical composition prepared with an amount of radionuclide that is suitable for multidose industrial scale production e.g., 50 MBq to 100 GBq.

Thus, the compositions of the present invention can be prepared with an amount of radionuclide that is 1 kBq to 10 GBq per dosing or with an amount of radionuclide that is 50 MBq to 100 GBq suitable for multidose industrial scale production.

The pharmaceutical composition of the present invention Infusion by the suspension with or without after-flushing using a pharmaceutical acceptable solution to obtain relevant distribution. The suspension of alpha emitter-labeled microparticles in a pharmaceutical composition can contain 50-3000 mg, such as 500-1500 mg for intraperitoneal infusion, and with an activity of 1 MBq-500 MBq, such as 4-12 MBq and dispersed in 1-2000 ml such as 10-500 ml such as 100-300 ml.

An aspect of the present invention relates to the use of the pharmaceutical composition of the present invention to infuse into the peritoneal cavity of a patient recently treated by cytoreductive surgery with or without HIPEC. The use and administration of alpha particle emitting radionuclides, e.g. in particles, following cytoreductive surgery of peritoneal tumors delivering a radiation equivalent dose range of 30 Sv to 10 000 Sv such as 50-1000 Sv, such as 100-500 Sv to the peritoneal surfaces. In one or more embodiments of the invention, the alpha emitting radionuclides is administered with a radiation dose range of 10 Gy (50 Sv) to 200 Gy (1 000 Sv). In one or more embodiments of the invention, the alpha emitting radionuclides is administered with a radiation dose range of 10 Gy (50 Sv) to 1000 Gy (5000 Sv). In one or more embodiments of the invention, the alpha emitting radionuclides is administered with a radiation dose range of 20 Gy (100 Sv) to 500 Gy (2500 Sv). In one or more embodiments of the invention, the alpha emitting radionuclides is administered with a radiation dose range of 10 Gy (50 Sv) to 2000 Gy (10 000 Sv). In one or more embodiments of the invention, the alpha emitting radionuclides is administered with a radiation dose range of 20 Gy (100 Sv) to 200 Gy (1000 Sv) In one or more embodiments of the invention, the alpha emitting radionuclides is administered with a radiation dose range of 10 Gy (50 Sv) to 500 Gy (2500 Sv). In one or more embodiments of the invention, the alpha emitting radionuclides is administered with a radiation dose range of 10 Gy (50 Sv) to 300 Gy (1500 Sv). The particle can have an average numerical diameter size of 0.1-30 μm, such as 1-10 μm, radiolabelled with 1-2000 atoms of alpha emitter per particle, such as 50-1000 atoms per particle.

In one or more embodiment of the present invention the particle can have an average numerical diameter size of 0.1-30 μm. In one or more embodiment of the present invention the particle can have an average numerical diameter size of 1-10 μm. In one or more embodiment of the present invention the particle can have an average numerical diameter size of 0.1-20 μm. In one or more embodiment of the present invention the particle can have an average numerical diameter size of 5-30 μm. To ensure appropriate antitumor effects and tolerable toxicity outcomes, activity levels used could be in the range of 2 MBq to 50 MBq of ²²⁴Ra per gram of microparticles, such as 10 MBq per gram of microparticles. To obtain relevant distribution vs peritoneal surface area and volumes, 0.3-5 gram of particle may be used as carrier for radiolabel, such as 0.7 gram of particle per dosing. In one or more embodiment of the present invention 0.3-5 gram of particles is used per dosing. In one or more embodiment of the present invention 0.7-1 gram of particles is used per dosing. In one or more embodiment of the present invention 0.1-10 gram of particles is used per dosing. In one or more embodiment of the present invention 0.5-5 gram of particles is used per dosing. In one or more embodiment of the present invention, the number of radioactive atoms can be in the range of 10-2000 atoms per particle, such as 50-1000 atoms per particle, such as 100-500 atoms per particle, such as 100-150 atoms per particle. In one or more embodiment of the present invention, the number of radioactive atoms can be in the range of 50-1000 atoms per particle. In one or more embodiment of the present invention, the number of radioactive atoms can be in the range of 100-150 atoms per particle. In one or more embodiment of the present invention, the number of radioactive atoms can be in the range of 50-500 atoms per particle. In one or more embodiment of the present invention, the number of radioactive atoms can be in the range of 50-200 atoms per particle. In one or more embodiment of the present invention, the number of radioactive atoms can be in the range of 50-1000 atoms per particle. Particles may be surface labelled, inclusion labeled, or first surface labeled followed by an outer layer of material to better trap the radionuclide(s).

Thus, and aspect of the present invention relates to a particle as defined herein of numerical diameter size 0.1-30 μm, such as 1-10 μm, radiolabelled with 1-2000 atoms of alpha emitter per particle, such as 50-500 atoms per particle. The alpha emitter can be ²²⁴Ra and can be dosage, and can be administered, as a suspension using a radioactivity dosage of 1-50 MBq, such as 4-20 MBq, such as 5-10 MBq per patient. This suspension can be a pharmaceutical composition as defined herein.

A further aspect of the present invention relates to a pharmaceutical composition, such as a suspension, comprising alpha emitter-labeled particles, that may be crystalline, containing 50-3000 mg such as 500-1500 mg of particles for intraperitoneal infusion, and with an activity of 1 MBq-500 MBq, such as 4-12 MBq, and dispersed in 1-2000 ml such as 10-500 ml such as 100-300 ml.

These particles, suspension and pharmaceutical compositions can be used in the treatment of cancer. The cancer can be tumors, and specifically intraperitoneal tumors. The administration can be local administration or infusion.

In one or more embodiments of the invention, the alpha emitting radionuclides is dosaged, and can be administered, with a radiation dose range of 30 Sv to 10000 Sv, more specifically 50-1000 Sv. The alpha emitting radionuclides can be administered with a radiation dose range of 200 Sv to 1000 Sv. The alpha emitting radionuclides can be administered with a radiation dose range of 200 Sv to 5000 Sv. The alpha emitting radionuclides can be administered with a radiation dose range of 100 Sv to 1000 Sv. The alpha emitting radionuclides can be administered with a radiation dose range of 300 Sv to 2000 Sv. The alpha emitting radionuclides can be administered with a radiation dose range of 100 Sv to 5000 Sv. The alpha emitting radionuclides can be administered with a radiation dose range of 50 Sv to 3000 Sv. The alpha emitting radionuclides can be administered with a radiation dose range of 50 Sv to 5000 Sv. The alpha emitting radionuclides can be administered with a radiation dose range of 30 Sv to 1000 Sv. The alpha emitting radionuclides can be administered with a radiation dose range of 30 Sv to 5000 Sv. The alpha emitting radionuclides can be administered with a radiation dose range of 50 Sv to 8000 Sv.

In one or more embodiments of the invention, the concentrations of phosphonates and or phosphate compounds are 1 microgram to 1000 milligram per ml, such as 0.1 mg to 10 mg per ml of final solution, or 1 microgram to 1000 milligram per gram particles in the final solution.

The particles can have a variety of characteristics, and the size of the particles can vary depending on the intended uses and applications.

The type of crystals may be any known form of degradable compound and sizes varying from 1 nm to 500 μm may be used. The size can be in the range of 100 nm to 50 μm and further preferably is size in the range of 1-10 μm. In one preferred embodiment is the size 1-10 μm. In another preferred embodiment the size is 100 nm to 5 μm, and in another 10-100 nm. In another preferred embodiment, the size is 1-20 μm, and in another 2-10 μm. The size of particles can be in the range of 0.5-30 μm, such as 1-10 μm, such as 1-4 μm. Median particle size in the range of 0.5-30 μm would cause suitable local retention.

An aspect relates to a composition comprising one or more particles according to the present invention. The composition may be a particle suspension comprising monodisperse or polydisperse particles comprising a degradable compound, a radionuclide and a phosphorus additive.

One or more embodiments of the present invention relates to the use of the particles of the present invention, where the radionuclide is either surface labeled by the radionuclide, inclusion labeled as part of particle volume, or a surface labeled particle that after radiolabeling is covered with a layer of material to protect the radiolabeled surfaces and prevent radionuclide release. The particle of the present invention can then become a radionuclide labeled particle whereby a layer of material has been added to cover the original surface to encapsulate the radionuclide.

The surface labelling can be performed as an adsorption of the radionuclide to the crystal particles driven by the affinity of the elements or the labelling can be performed as co-precipitation where additional inorganic compounds aid the precipitation process. A chelator can be use in this process, and the chelator can be incorporated in the particle or on the surface of the particle.

An aspect of the present invention relates to a composition comprising a particle comprising a degradable compound and a radionuclide, wherein a phosphorus containing additive is comprised in the composition. The composition can be a suspension of particles. The phosphorus containing additive can be incorporated into the particle. The phosphorus containing additive can be associated with the surface of the particle or be present in the surroundings of the particle, i.e. in the composition or suspension that the particle is part of. Thus, one aspect of the present invention relates to a composition or suspension comprising a particle, wherein the particle comprises a degradable compound, a radionuclide and a phosphorus containing additive, and wherein the phosphorus containing additive is associated with the particle by being present in the composition or suspension. The phosphorus containing additive can be as part of the particle. The presence can be on the surface of the particle. The presence can be as part of the composition or suspension of particles. The presence can also be as part of the particle and as part of the composition or suspension of particles.

One or more embodiments of the present invention relate to a particle suspension which is a mixture of a solid phase and a liquid phase. The phosphorus containing additive may either be in the liquid phase. The containing phosphorus additive can be in the solid phase. The phosphorus containing additive can be the solid and the liquid phases. In the solid phase the phosphorus containing additive can be on the surface or embedded in the particles or both on the surface or embedded in the solid phase. The solid phase might be made out of nanoparticles, microparticles or a combination those two. The radionuclide may be associated with the surface of the particle or embedded in the volume or bulk of the particle, or both. The solid phase can therefore comprise a particle comprising a degradable compound and a radionuclide, with or without a phosphorus containing additive, but the phosphorus containing additive will always be in the liquid phase if it is not part of the solid phase. The degradable compound, radionuclide and phosphorus containing additive can be any of those disclosed herein.

The phosphorus containing compound may or may not complex radionuclide.

The composition of the present invention is preferably an aqueous composition. Thus, in this embodiment the liquid phase is an aqueous phase. The composition can be a saline composition.

The composition can also be an alcohol composition. The composition can be a gel-matrix composition. The composition of the present invention can be a suspension of the particles of the present invention.

Thus, a further aspect of the present invention relates to a composition or a pharmaceutical composition comprising one or more particles according to the invention and a diluent, carrier, surfactant, deflocculant and/or excipient. In one or more embodiments of the invention, the pharmaceutical composition is comprising one or more particles according to the present invention and a diluent, carrier, surfactant, and/or excipient.

Acceptable carriers and pharmaceutical carriers include but are not limited to non-toxic buffers, fillers, isotonic solutions, solvents and co-solvents, anti-microbial preservatives, anti-oxidants, wetting agents, antifoaming agents and thickening agents etc. More specifically, the pharmaceutical carrier can be but are not limited to normal saline (0.9%), half-normal saline, Ringer's lactate, dissolved sucrose, dextrose, e.g. 3.3% Dextrose/0.3% Saline. The physiologically acceptable carrier can contain a radiolytic stabilizer, e.g. ascorbic acid, human serum albumin, which protect the integrity of the radiopharmaceutical during storage and shipment.

The particles may be dispersed in various buffers compatible with medical injections, e.g., dissolved salts and/or proteins and/or lipids and or sugars.

The pharmaceutical compositions can comprise a multitude of particles. These can be the same or different.

Thus, in one or more embodiments of the invention, the size of the particle is from 1 nm to 500 μm.

The particles and compositions or the present invention can be used as radiotherapeutic compounds and/or radiotherapeutic mixtures (compositions and solutions).

An aspect of the invention relates to the particle, composition or pharmaceutical composition of the present invention for use as a medicament. An aspect of the invention relates to the particle, composition or pharmaceutical composition of the present invention for use in the treatment of cancer.

Intracavitary therapy may include treatment of e.g., intraperitoneal cancers, intracranial cancers, pleural cancers, bladder cancers, cardiac cancers, cancers in the subarachnoid cavity. Examples of cavities where the particles may be used is cranial cavity, thoracic cavity, lung cavity, spinal cavity, pelvic cavity, pericardium, pleural cavity, bladder cavity or a combination of these including cancers spreading on the peritoneum or meninges and organs within any of these cavities.

In one or more embodiments of the invention, the cancer is selected from the group consisting of intraperitoneal cancers, intracranial cancers, pleural cancers, bladder cancers, cardiac cancers, cancers in the subarachnoid cavity, and non-cavitary targets such as melanoma, non-small-cell-lung cancer, and metastasis.

In one embodiment of the present invention is the cancer selected from the group consisting of metastatic cancer, lung cancer, ovarian cancer, colorectal cancer, stomach cancer, pancreatic cancer, breast cancer, neoplastic meningitis, peritoneal cancer, pleural effusion, malignant mesothelioma, breast cancer, sarcomas, brain cancers like glioblastoma and astrocytoma, prostate cancer, bladder cancer, and liver cancer. One or more embodiments of the present invention relates to the use according to the invention, wherein the cancer is metastatic cancer. One or more embodiments of the present invention relates to the use according to the invention, wherein the cancer is lung cancer. One or more embodiments of the present invention relates to the use according to the invention, wherein the cancer is ovarian cancer. One or more embodiments of the present invention relates to the use according to the invention, wherein the cancer is colorectal cancer. One or more embodiments of the present invention relates to the use according to the invention, wherein the cancer is stomach cancer. One or more embodiments of the present invention relates to the use according to the invention, wherein the cancer is pancreatic cancer. One or more embodiments of the present invention relates to the use according to the invention, wherein the cancer is breast cancer. One or more embodiments of the present invention relates to the use according to the invention, wherein the cancer is neoplastic meningitis. One or more embodiments of the present invention relates to the use according to the invention, wherein the cancer is peritoneal cancer. One or more embodiments of the present invention relates to the use according to the invention, wherein the cancer is pleural effusion. One or more embodiments of the present invention relates to the use according to the invention, wherein the cancer is pleural effusion. One or more embodiments of the present invention relates to the use according to the invention, wherein the cancer is malignant mesothelioma. One or more embodiments of the present invention relates to the use according to the invention, wherein the cancer is breast cancer. One or more embodiments of the present invention relates to the use according to the invention, wherein the cancer is prostate cancer. One or more embodiments of the present invention relates to the use according to the invention, wherein the cancer is pericardial cancer One or more embodiments of the present invention relates to the use according to the invention, wherein the cancer is sarcoma. One or more embodiments of the present invention relates to the use according to the invention, wherein the cancer is brain cancers like glioblastoma and astrocytoma. One or more embodiments of the present invention relates to the use according to the invention, wherein the cancer is bladder cancer. One or more embodiments of the present invention relates to the use according to the invention, wherein the cancer is liver cancer. One or more embodiments of the present invention relates to the use according to the invention, wherein the cancer is micrometastasis, which can be intraperitoneal.

The pharmaceutical composition of the present invention, such as Radspherin, may be used after surgery in cancers where peritoneal spread of cancers cells may be a problem including Adrenal cancer, Appendix cancer, Colon and rectal cancer, Gastric (stomach) cancer, Liver cancer, Mesothelioma, Ovarian cancer, Pancreatic cancer, Peritoneal cancer. The cancer may be Adrenal cancer. The cancer may be Appendix cancer. The cancer may be Colon and rectal cancer. The cancer may be Gastric (stomach) cancer. The cancer may be Mesothelioma. The cancer may be Pancreatic cancer. The cancer may be Peritoneal cancer.

In a special embodiment for the use of the particles of the present is treatment or amelioration of a disease which is an infection or inflammation rather than or in combination with cancer. The inflammation can for example be arthritis.

In one embodiment of the present invention is the infection selected from the group consisting of a bacterial infection and viral infection.

Radioembolization may include treatment of primary or metastatic cancer in an organ e.g., the liver by administering the particles of the present invention to a blood vessel leading to a tumor in the liver or another solid organ infiltrated by tumor tissue.

Radiosynovectomy for joint disorders including chronic inflammations is targeted radiation treatment for painful joint diseases using radioactive substances. Its use includes treatment of hemophilic arthritis.

The particles are preferably administered by local injection, e.g. intracavitary. In a special embodiment the particles are injected directly into a tumor.

Another aspect of the present invention relates to a method of treatment, inhibition, or amelioration comprising administration of the particles or the pharmaceutical composition of the present invention to an individual in need thereof, as described herein.

In one or more embodiments of the invention, the cytoreductive surgery of one or more peritoneal tumor(s) is performed preoperatively, e.g., the same day, one day prior to the administration of the pharmaceutical composition comprising a therapeutically relevant amount of alpha emitting radionuclides, such as at least two days, such as at least three days, such as at least four days, such as at least five days, such as at least six days, such as at least seven days, such as at least eight days, such as at least nine days, such as at least ten days, such as at least eleven days, such as at least twelve days, such as at least thirteen days, such as at least fourteen days. In one or more embodiments of the invention, the cytoreductive surgery of one or more peritoneal tumor(s) is performed preoperatively, e.g., the same day the administration of the pharmaceutical composition comprising a therapeutically relevant amount of alpha emitting radionuclides, as described herein. In one or more embodiments of the invention, the cytoreductive surgery of one or more peritoneal tumor(s) is performed one day prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed one day prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed two days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed three days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed four days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed five days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed six days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed seven days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed eight days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed nine days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed ten days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed eleven days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed twelve days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed thirteen days prior to the administration of the pharmaceutical composition comprising a therapeutically relevant amount of alpha emitting radionuclides, as described herein. In one or more embodiments of the invention, the cytoreductive surgery of one or more peritoneal tumor(s) is performed fourteen days prior to the administration of the pharmaceutical composition comprising a therapeutically relevant amount of alpha emitting radionuclides, as described herein.

In one or more embodiments of the invention, the pharmaceutical composition is a medical device or is comprised in a medical device.

A medical device is any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings for the purpose of: Diagnosis, prevention, monitoring, treatment or alleviation of disease; Diagnosis, monitoring, treatment, alleviation of or compensation for an injury or handicap; Investigation, replacement or modification of the anatomy or of a physiological process; Control of conception; and which does not achieve its principal intended action in or on the human body by pharmacological, immunological or metabolic means, but which may be assisted in its function by such means Medical devices vary according to their intended use and indications. Examples range from simple devices such as tongue depressors, medical thermometers, and disposable gloves to advanced devices such as computers which assist in the conduct of medical testing, implants, and prostheses.

According to the FDA is medical device “an instrument, apparatus, implement, machine, contrivance, implant, in vitro reagent, or other similar or related article, including a component part, or accessory which is: recognized in the official National Formulary, or the United States Pharmacopoeia, or any supplement to them, intended for use in the diagnosis of disease or other conditions, or in the cure, mitigation, treatment, or prevention of disease, in man or other animals, or intended to affect the structure or any function of the body of man or other animals, and which does not achieve any of its primary intended purposes through chemical action within or on the body of man or other animals and which is not dependent upon being metabolized for the achievement of any of its primary intended purposes.”

The present particles are not being metabolized nor do they have significant chemical action within the body. The particles are carriers of radioactivity that are designed not be metabolized or have any chemical action within the body, and this allows for radiotherapy with very limited unwanted side-effects, such as toxicity.

Thus, in one embodiment is the term “medical device” understood as FDAs definition above.

The degradable particle can comprise many different additional compounds. These can serve various purposes included targeting, stability, solubility and rate of degradation.

In one embodiment of the present invention, the particle comprises one or more compounds selected from the group consisting of a monoclonal antibody, a polyclonal antibody, a radioimmunoconjugate, an immunoconjugate, a chelate antibody conjugate, vitamins including folate and folate derivatives, peptides, minibodies, and affibodies.

In an embodiment, the particles comprise antibody, antibody fragment or protein or peptide or vitamin derivative (targeting conjugate) with affinity for receptors including antigens on the tumor cells.

In another embodiment, the particles comprise radiolabeled antibody, antibody fragment or protein or peptide or vitamin derivative (targeting conjugate) with affinity for receptors including antigens on the tumor cells whereby the labeled particles will give a general particle radiation field on the surfaces, and the labeled antibody or similar gives a specific alpha particle dose to the tumor cells by receptor or antigen binding.

The radionuclides in the present invention can be conjugated to a targeting molecule by using bifunctional chelators.

These could be cyclic, linear or branched chelators. Particular reference may be made to the polyaminopolyacid chelators which comprise a linear, cyclic or branched polyazaalkane backbone with acidic (e.g. carboxyalkyi) groups attached at backbone nitrogens.

Examples of suitable chelators include DOTA derivatives such as p-isothiocyanatobenzyl-1,4,7,10-tetraazacyclododecane-1,4,7,10-tetraacetic acid (p-SCN-Bz-DOTA) and the tetra primary amide variant of this DOTA compound, termed TCMC, and DTPA derivatives such as p-isothiocyanatobenzyl-diethylenetriaminepenta-acetic acid (p-SCN-Bz-DTPA), the first being cyclic chelators, the latter linear chelators.

Metallation of the complexing moiety may be performed before or after conjugation of the complexing moiety to the targeting moiety.

The radiolabeling procedure will in general be more convenient in terms of time used etc. if the chelator is conjugated to the antibody before the radiolabeling takes place.

An aspect of the invention relates to the particle, composition or pharmaceutical composition of the present invention for use according to the present invention, which is used in combination with other cancer therapies.

Examples of therapies are chemotherapy like taxanes (e.g. paclitaxel, docetaxel), platins (e.g. carboplatin, cisplatin), doxorubicin, mitomycin. Other examples are DNA repair inhibitors, such as PARP inhibitors (e.g. Olaparib, Rucaparib, Niraparib, Talazoparib, Veliparib, Pamiparib, CEP 9722, E7016, and 3-Aminobenzamide). Further examples are radioimmunotherapies.

The DNA repair inhibitor can be selected from the group consisting of a poly (ADP-ribose) polymerase inhibitor (PARPi), a MGMT inhibitor, a DNA-dependent protein kinase inhibitor (DNA-PK inhibitor), a ataxia telangiectasia and Rad3-related (ATR) kinase inhibitor, a ataxia telangiectasia mutated (ATM) kinase inhibitor, a Wee1 kinase inhibitor, and a checkpoint kinase 1 and 2 (CHK1/2) inhibitor.

In one or more embodiments of the present invention, the PARPi is selected from the group consisting of Olaparib, Rucaparib, Niraparib, Talazoparib, Veliparib, Pamiparib, CEP 9722, E7016, and 3-Aminobenzamide.

The PARPi can be Olaparib. The PARPi can be Rucaparib. The PARPi can be Niraparib. The PARPi can be Talazoparib. The PARPi can be Veliparib. The PARPi can be Pamiparib. The PARPi can be CEP 9722. The PARPi can be E7016. The PARPi can be 3-Aminobenzamide.

### Method for Preparing the Particles

An aspect of the invention relates to a method for preparing a particle according to the present invention, the method comprising bringing a degradable compound, a radionuclide, and a phosphorus containing additive in contact with each other with or without using a carrier for the radionuclide. A degradable compound and a radionuclide can form a first particle in an initial step, which can be followed by an additional step of adding a degradable compound to the already radiolabeled particle, thereby giving a layered particle. Subsequently a phosphorus containing additive is added to stabilize the particle. The phosphorus containing additive can in this process become part of the particle and remain in the composition where the particle is. The phosphorus containing additive can also both be part of the particle and remain in the composition comprising the particle.

Phosphorus containing compounds such as phosphonates and phosphates can be applied as additives to stabilize crystal particles, i.e. they are the phosphorus containing additives described herein. They can be added to particles, such as crystal particles, during the formation of these, after the particles have been formed, after labeling or to the final formulation in order to achieve the size control. The product can be sterilized by autoclaving and the additives can be included before or after this process. The size controlling additive can also be used as a component of a kit used to prepare a final product.

Thus, in embodiments an additional size controlling additive is added in the preparation of the particle. In embodiments the additional size controlling additive is an alcohol such as but not limited to a diol or triol. In embodiments the additional size controlling additive is selected from the group consisting of Etylene glycol (EG), PEG, Glycerol and Dextran, Ethanol. In embodiments the additional size controlling additive is Glycerol.

The phosphorus containing compounds can stabilize crystal particles of monodisperse and polydisperse particles. The particles will typically be polydisperse because they are made in solution, but they can share characteristics, such as having a size within a given similar range.

Thus, an aspect of the present invention relates to the use of a phosphorus containing additive for size control of particles comprising a degradable compound. Another aspect of the present invention relates to the use of a phosphorus containing additive for stabilizing particles comprising a degradable compound. These particles can be the particles of the present invention, before or after a radionuclide is added. The particles can therefore be the degradable compound itself. In one embodiment of the present invention, the degradable compound is a crystal particle.

A solution or composition comprising radionuclide, e.g. a ²²⁴Ra solution or composition with progeny ²¹²Pb in mixture could be pretreated with chelate-antibody conjugate to complex ²¹²Pb prior to particle labeling to produce a two-component therapeutic system containing a radioimmunoconjugate for ²¹²Pb antigen-specific treatment and alpha emitter, e.g. ²²⁴Ra-labeled particles for a general cavity treatment. A biologic compound, such as an antibody, can also be part of these particles. These can then subsequently be mixed with a phosphorus containing additive to give a size controlled particle. The particle of the composition could also be ²¹²Pb-labeled particles in a composition with a ²¹²Pb antigen-specific treatment.

An embodiment relates to a three-component system or kit comprising a radionuclide such as a radioimmunoconjugate for antigen-specific treatment, a degradable compound, and a phosphorus containing additive.

The preferable way to use this would be by a kit containing a vial A with chelate-conjugated antibody and a vial B with radionuclide, e.g. ²²⁴Ra in equilibrium with daughter nuclides, and a vial C with microparticles, whereby the content of A is added to vial B, or vice versa, and incubated from a few minutes to a few hours before the mixture is transferred to vial C for further incubation for a few minutes to a few hours before being mixed with the phosphorus containing additive and subsequently transferred to a syringe and injected into the patient.

This principle could significantly reduce the level of for example ²¹²Pb-radioimmunoconjugate needed for therapy since ²²⁴Ra-particles is expected to contribute strongly to the antitumor activity in such a system.

Another aspect of the present invention relates to a kit comprising a nano- or micro-particle according to the present invention, and optionally instructions to use the kit.

In one embodiment of the present invention, the kit comprises a chelator-conjugated molecule, including monoclonal antibody.

The current methods and product allow for centralized production and shipment to the end user since the radionuclide has several days half-life. Another aspect of the presented invention is the use of a biodegradable particle that slowly dissolves into calcium and carbonate thereby producing small amounts of products that are already abundantly present in the body. It is also noteworthy of the following feature: When a radionuclide, e.g. ²²⁴Ra is absorbed the degradable compound, such as calcium carbonate, there is a significant release of short living ²²⁰Rn (t_{1/2}=56 s) which will together with the ultra-short lived ²¹⁶Po (t_{1/2}=0.16 s) produce two alpha particles before decaying to the longer lived beta emitter ²¹²Pb (t_{1/2}=10.6 h). Lead has a very high precipitability with for example calcium carbonate so the ²¹²Pb in the i.p. fluid will tend to re-associate to the particles diminishing leakage of ²¹²Pb into the systemic circulation.

It is therefore a very special technical feature that ²²⁴Ra decays into a gas that can diffuse out of the particle and afterwards decay further into ²¹²Pb that precipitates with calcium carbonate.

Pre-produced particles and subsequent surface sedimentation or radionuclide co-sedimentation for deeper inclusion of radionuclide are two methods useful for producing a therapeutic product. Once the radionuclide and the degradable compound has formed a first particle, the phosphorus containing additive can subsequent size control the first particle and become the particle of the present invention.

The particles may be produced in sizes from nanometers to several tens of micrometers and radiolabeled with high labeling yields and can be stored for several days which is important since it allows centralized production and shipment to the hospitals of ready to use particle suspensions. This can be done without risking the particle integrity and therefore ensure that the particles remain intact and have the size that is needed for the intended use.

As aspect of the present invention relates to a particle produced by any of the methods described herein. The particle can be a microparticle.

### Some Definitions

CRC: Colorectal cancer.

EDTMP: Ethylenediamine tetra(methylene phosphonic acid) or salts thereof.

Cytoreductive surgery (CRS): The surgical removal of visible cancerous tumors from the abdominal cavity. Methods are used with or without concomitant HIPEC therapy.

Equivalent dose: Equivalent dose is a measure of the radiation dose to tissue where a correction has been made for the RBE to allow for the different relative biological effects of different types of ionizing radiation. In quantitative terms, equivalent dose is less fundamental than absorbed dose, but it is more biologically significant. Equivalent dose is measured using the sievert (Sv) HIPEC: Hyperthermic intraperitoneal chemotherapy (HIPEC) is a method to wash the peritoneal cavity with heated chemotherapy in an effort to inactivate residual cancer cells after CRS.

I.p.: intraperitoneal

LET: Linear energy transfer, Linear energy transfer (LET) is the average energy deposited per unit length of track, and describes the pattern of energy deposition within a photon or particle track. Conventional therapeutic radiation is sparsely ionizing and considered low-LET radiation. High LET radiation includes particles with substantial mass and charge such as alpha particles. Low energy neutrons, which carry no electrical charge, are also a high-LET radiation. The distribution of energy in cells has a marked influence on the amount of biological damage done by a fixed amount of radiation.

Microparticle diameter: The numerical median diameter of particles in a suspension, which is roughly approximately equal to ⅓ of the volumetric mean diameter of the particles in the suspension.

Monodisperse: Having particles of approximately the same size.

OC: ovarian cancer.

Polydisperse: Having a range of particle size.

Radspherin: microparticles made up of calcium carbonate radiolabelled with ²²⁴Ra and used as a suspension.

In the text, micro-meter may be written as um or μm.

RBE: Relative biological effectiveness (RBE) is a relative measure of the damage done by a given type of radiation per unit of energy deposited in biological tissues. Alpha particles, which is high-LET, has a high RBE versus low-LET beta-particles, X-rays and external radiation therapy. An RBE of 5 for alpha particles vs low LET radiation is often assumed for therapeutic levels of radiation (An RBE of 20 for alpha particles is commonly assumed for radiation protection purposes regarding low level exposure of a population).

XBR: External beam radiotherapy.

### General

It should be understood that any feature and/or aspect discussed above in connections with the compounds according to the invention apply by analogy to the methods described herein.

The following figures and examples are provided below to illustrate the present invention. They are intended to be illustrative and are not to be construed as limiting in any way.

### BRIEF DESCRIPTION OF THE FIGURES

FIG. 1 shows gamma camera scans of the peritoneal cavity of a patient at 3 h (left), 26 h (middle) and 50 h (right) after the local administration of Radspherin to the peritoneal cavity.

### EXAMPLES

### Example 1—Treatment of Patients with Colorectal Cancer with Surgery+HIPEC+Radspherin

Methods: patients were treated with cytoreductive surgery (CRS) and hyperthermic intraperitoneal chemotherapy HIPEC and at day 2-4 after the surgery received Radspherin infusion via a catheter into the peritoneal cavity. The patients were typically discharged within one week after the surgery.

In the phase I dose escalation part of the study the Radspherin dosing of 1 MBq, 2 MBq 4 MBq and 7 MBq were used with at least three patients on each dose step to look for dose limiting toxicity. Gamma camera imaging were performed in a subset of the patients. The patients were followed for recurrences using standard radiological methods.

### Example 2—Adverse Event Frequency in Colorectal Cancer Patients Treated by Cytoreductive Surgery Followed by HIPEC and Radspherin

Method: The data for CRS+HIPEC+Radspherin treated patients are compared to historical control data of CRS+HIPEC from similar patients from same institution (Froysnes et al, 2016) in Table 1.

Results: The data in table 1 shows that the percent of serious adverse events grade III or worse was not increased by the addition of Radspherin to the therapeutic combination neither at lower dosages nor at the highest dosage. The percent were 15%, 9% and 8% for CRS+HIPEC historical control, CRS+HIPEC+1-4 MBq Radspherin and CRS+HIPEC+7 MBq Radspherin groups respectively.

In conclusion, there were no indications that a dosage of 7 MBq Radsperin given after CRS+HIPEC caused increased local toxicity in terms of serious adverse events.

TABLE 1
Serious adverse events (SAE) grade III or higher in colorectal cancer
patients treated with CRS + HIPEC with or without Radspherin.

| | Number of patients (%) | | Physicians comments about |
|---|---|---|---|
| | with one or more SAE | | likelihood of related to |
| Treatment group | grade 3 or higher | | Radspherin |
| Historical control CRS + HIPEC | 18/119 | (15%) | |
| CRS + HIPEC + Radspherin 1-4 MBq | 1/11 | (9%) | Unlikely |
| CRS + HIPEC + Radspherin 7 MBq | 1/12 | (8%) | Unlikely |

### Example 3—Treatment of Patients with Ovarian Cancer with Surgery+Radspherin

Methods: patients were treated with cytoreductive surgery (CRS) (but not HIPEC) and at day 2-4 after the surgery received Radspherin infusion via a catheter into the peritoneal cavity. The patients were typically discharged within one week after the surgery.

In the phase I dose escalation part of the study the Radspherin dosing of 1 MBq, 2 MBq 4 MBq and 7 MBq were used with at least three patients on each dose step to look for dose limiting toxicity. Gamma camera imaging were performed in a subset of the patients. The patients were followed for recurrence by standard radiological methods.

### Example 4—Simplified Peritoneal Dosimetry for Radspherin Alpha Particle Therapeutic

Background: ²²⁴Ra-labeled microparticles (Radsperin) was administered by intraperitoneal infusion to patients following cytoreductive surgery and the activity level determined by gamma camera scan at different time points after infusion. Table 3 presents the different alpha particles produced in the ²²⁴Ra series constituting the radiation source of Radspherin. Each radium atom will produce in total 4 alpha particles when decaying via the progeny nuclides.

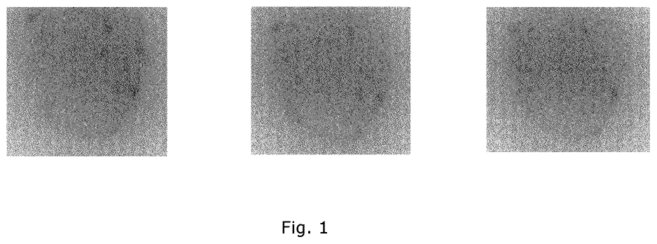

Based on the signals measured with the gamma camera scans (e.g., FIG. 1) of patients that had received Radspherin, it was estimated that on average approximately 75% of the administered activity would decay in the peritoneal cavity.

TABLE 3
Radionuclides in Radspherin: Alpha particle ranges
calculated by the use of the NIST app ASTAR.

| Radionuclide (half-life) | Alpha energy (MeV) | Range in water (μm) |
|---|---|---|
| ²²⁴Ra (3.6 days) | 5.7 | 46 |
| ²²⁰Rn (56 s.) | 6.3 | 53 |
| ²¹⁶Po (0.15 s.) | 6.8 | 60 |
| ²¹²Bi [36.2%] (1 h) | 6.1 | 50 |
| ²¹²Po [63.8%] (0.3 us.) | 8.8 | 90 |

### Dosimetry Calculations.

In the following work a quality factor of 5 for alpha particles vs XBR was assumed yielding 5 Sv per Gy at therapeutic level of alpha particle radiation. The calculations are done for the highest dosing tested of Radpsherin, i.e., 7 MBq per patient.

### Part 1:

Assumption: Radiolabeled product distributed mainly as a uniform sheet covering surfaces with the same specific activity in the liquid and on the surfaces. The tissue surfaces are covered with a 35 μm liquid film.

Assuming the uniform dose on both sides of the “particle sheet” within the “radiation zone” of 35 μm on each side of the “particle sheet”.
Assumption: Peritoneal surface area=17500 cm²
Peritoneal retention factor of injected radionuclide: 75%
Average thickness of the radiation zone on surfaces: 35 μm×2=70 μm>122.5 ml volume of surface areas irradiated (half of this would be the liquid film on the surface and half of it would be tissue) (i.e. Surface Region).

$$1.6 \times {10}^{13} J / MeV$$

27.3 MeV alpha particles from ²²⁴Ra series

Using this model the radiation dose would be as follow:

$$Dose = ( 7 \times {10}^{∧} 6 \times 3600 \times 24 \times 3.6 \times 0.75 \times 1.6 \times {10}^{∧} - 13 \times 27.5 ) / ( ln 2 \times 0.1225 ) = 84.6 Gy \to 423 Sv ( 5 Sv / Gy )$$

### Part 2: Uniform Liquid Distribution of Radioactivity

Peritoneal liquid volume 75-500 ml
   If peritoneal liquid volume is on average 75 ml (normal person), the total irradiation volume (including surface tissue)
would be 122.5/2+75 (includes the liquid film)=136.25 ml
   If peritoneal liquid volume is on average 500 ml, the total irradiation volume would be 122.5/2+500 (includes liquid film)=561.25 ml
Calculations are for 7 MBq ²²⁴Ra in Radspherin
   75 ml liquid volume:

$$Dose = ( 7 \times {10}^{∧} 6 \times 3600 \times 24 \times 3.6 \times 0.75 \times 1.6 \times {10}^{∧} - 13 \times 27.5 ) / ( ln 2 \times 0.13625 ) = 75.5 Gy \to 378 Sv ( 5 Sv / Gy )$$

500 ml liquid volume:

$$Dose = ( 7 \times {10}^{∧} 6 \times 3600 \times 24 \times 3.6 \times 0.75 \times 1.6 \times {10}^{∧} - 13 \times 27.3 ) / ( ln 2 \times 0.56125 ) = 18.4 Gy \to 92 Sv ( 5 Sv / Gy )$$

Assuming a volume of 200 ml liquid as the average, this would result in an equivalent dose to the liquid of 198 Sv according to this calculation model.

### Example 5—Recurrence Free Survival after Standard Treatment (Previously Published) Versus Standard Treatment+Radspherin Alpha-Particle Therapy in Second Line Colorectal Cancer Following Cytoreductive Surgery+HIPEC

Methods: Control group is colorectal cancer (CRC) patients with resectable disease receiving HIPEC following complete cytoreductive surgery (CRS).

In the control group the progression at 12 months was interpolated value from the published survival curve (Frøysnes et al., 2016). Control (C) and Radspherin (R) group similarities: Female 65% (C) 70% (R); Median Age 58 (C) 64 (R); HIPEC type mainly Mitomycin C in both groups (exept for the few patients receiving Oxaliplatin, i.e., 4/23 in the Radspherin group). The patients were followed for recurrence by standard radiological methods.

TABLE 4
CRC, CRS + HIPEC with or without Radspherin
at 12 months recurrence free survival

| | | |
|---|---|---|
| CRS + HIPEC + Radspherin 1- | 61% | N = 23 |
| 7 MBq | | |
| CRS + HIPEC + Radspherin 2- | 68% | N = 19 |
| 7 MBq | | |
| CRS + HIPEC + Radspherin 7 | 75% | N = 12 |
| MBq + w/w.o. repeated | | |
| CRS + HIPEC -Frøysnes et | 44% (interpolation) | N = 117 |
| al., 2016 | | |

Results: Table 4 shows that the % of recurrence free patients at 12 months was higher in cytoreductive surgery (CRS) with HIPEC+Radspherin treated patient group compared to the historical controls of CRS+HIPEC treatment without Radspherin.

I.e. for the CRS+HIPEC+7 MBq Radspherin group, the 12 months recurrence rate was reduced to less than half of that of the historical control group receiving CRS+HIPEC.

So far, data from the ongoing study for 15 months and 18 months follow up of the 7 MBq group indicate 0/8 and 0/6 of patients had local i.p. recurrence in the peritoneum at these time points.

In conclusion, the data so far indicate a substantial antitumor effect from the Radspherin treatment.

### Example 6—Recurrence Free Survival after Standard Treatment (Previously Published) Versus Standard Treatment+Radspherin Alpha-Particle Therapy in Ovarian Cancer 2. Line Following Cytoreductive Surgery

Methods: Second line ovarian cancer with macroscopic tumor removed by cytoreductive surgery received Radspherin by catheter infusion into the intraperitoneal cavity 2-4 days after completion of the surgery. The patients were followed for recurrence by standard radiological methods.

TABLE 5
CRS with or without Radspherin at
12 months recurrence free survival

| | | |
|---|---|---|
| Radspherin + CRS | 80% | N = 10 |
| CRS - Harter et al 2021 | 68% | N = 206 |
| CRS - Coleman et al 2019 | 63% | N = 245 |

Results: Table 5 shows current status. There are no i.p. metastases (only two distal) at the 12 month time point in the ovarian cancer groups receiving Radspherin following CRS (without HIPEC) which is a promising trend.

### Example 7—Metastasis Pattern in Patients Receiving Radspherin Following Cytoreductive Surgery for Colorectal Cancer and Ovarian Cancer

Methods: The metastases pattern in patients treated for peritoneal tumors with CTR (with or without HIPEC)+Radspherin is presented. Represent 12 months follow-up.

TABLE 6
Metastasis pattern after 12 months follow up of patients treated
with cytoreductive surgery followed by Radspherin alpha therapy.

| Disease (dose, MBq) | Pure i.p. | i.p. + distal | Total i.p. | Pure distal |
|---|---|---|---|---|
| CRC (1-2) | 2/7 | 1/7 | 3/7 | 1/7 |
| CRC (7) | 0/12 | 0/12 | 0/12 | 3/12 |
| OC (1-4) | 0/10 | 0/10 | 0/10 | 2/10* |

*Lymph node metastase in one of the patients with recurrence.

### Results

Colorectal cancer: Table 6 shows that after 12 months of follow-up for low dosages of 1-2 MBq per patients of Radspherin in colorectal patients there are observed i.p. metastases in 3 of 7 patients while at 7 MBq none (0/12) had developed i.p. metastases (Table 6). This indicates that the protective effect of Radspherin is dosage dependant and are in full effect at the 7 MBq dosage level.

Ovarian cancer: The limited amount of data presented are promising with no intraperitoneal metastases observed up to the 12 month follow-up.

### Example 8—Radiotherapy and Wound Healing

Radspherin has been presented previously in preclinical studies showing a relevant safety profile. The models used were healthy mice without wounds that was either not injected (safety) or injected with tumor cells (therapy) and subsequently with Radspherin to study safety and antitumor activity.

However, the preclinical models used do deviates from the clinical situation in the important aspect that they have no wounds while the patients have freshly made surgical wounds due to cytoreductive surgery before Radspherin administration.

It is known in the field that radiotherapy could affect wound healing negatively, especially at higher radiation doses (Diaz et al., Surgeries 2021, 2(1), 35-57).

Although radiation therapy is used to kill cancerous cells, it also damages healthy cells, leading to many acute and long-term side effects. One serious complication is problems with wound healing.

The long-term effects of radiotherapy include skin atrophy, soft tissue fibrosis and microvascular damage, leading to a higher risk of developing problematic, non healing wounds which are unamenable to surgical repair (Dormand et al., Int Wound J. 2005 June; 2(2): 112-127).

Complications after radiation therapy occur in up to 60 percent of surgical patients (Haubner et al., Radiat Oncol. 2012; 7: 162.).

Radiotherapy is an invaluable weapon when treating cancer. However, the deleterious effects of radiation, both immediate and long-term, may have a significant effect on local tissues. Problematic wound healing in radiation-damaged tissue constitutes a major problem. Poor wound healing may lead to chronic ulceration, pain, secondary infection and psychological distress and compromise the outcome of general or reconstructive surgery.

Also, alpha-particle irradiation has been reported to affect negatively the wound healing. E.g., in an experimental model (Xueting et al., Poster viewing Q&A session volume 105, issue 1, supplement, E661, Sep. 1, 2019, International Journal of Radiation Oncology), even at relative modest dose of 0.56 Gy (assuming 5 Sv/Gy equivalent dose of 2.8 Sv) wound healing was affected.

To our surprise it was found that at equivalent dose significantly above 100 Sv (assuming 5 Sv/Gy) in the intraperitoneal cavity with newly made surgical wounds from cytoreductive surgery there were no signs of complications from Radsperin treatment as indicated by no significant change in frequency of adverse events.

In a case whereby a patient by accident received an estimated 1 MBq of Radspherin (from a 7 MBq dosage) subcutaneously in the catheter incision due to extravasation caused by a sliding catheter, the catheter incision wound was healed after two months without ulceration or necrosis. This was unexpected since it is known from the literature that extravasation of radiopharmaceuticals can lead to severe soft tissue damages (Van der Pol et al, 2017). This demonstrates that Radspherin at clinical level also may have positive suitable toxicity profile with extravasation.

### Example 9—Comparative Dosimetry

Method: Dosimetry estimates for Radspherin were compared with the literature data for radiation treatment with external beam fractionation therapy and radionuclide therapy. Low linear energy transfer (LET) radiation from external beam radiation and beta emitter ³²P with radiobiological effectiveness (RBE)=1 compared with high LET alpha-radiation from 211At (+ progeny), ²¹²Pb (+ progenies) and ²²⁴Ra (+ progenies) with an estimated RBE=5.

TABLE 7
Comparison of various radiation sources used for peritoneal radiation treatment.

| Radiation source | Dose/equivalent dose | | |
|---|---|---|---|
| (RBE) | (Gy/Sv) | Comment | References |
| External beam (1) | 30/30 | Tolerable toxicity. 1.5 | Arians N et al., 2019. |
| | | Gy × 20 fractionation. | |
| | | Following CRS. | |
| 32P Phosphocol (1) | 51/51* | Some late toxicity. | Rxlist |
| | | Assuming surface | Watson EE et al., |
| | | deposition | 1989. |
| 211At-labeled | 3.35/16.8 | Tolerable toxicity. | Andersson H et al., |
| antibody (5) | | Peritoneum dose 15.6 | 2009. |
| | | mGy/(MBq/L). 215 | Hallqvist A et al., |
| | | MBq/patient max | 2019. |
| | | dosing | |
| ²¹²Pb-labeled | 3.5/17.5 | Tolerable toxicity. | Meredith R et al., |
| antibody (5) | | Approximately 55 | 2014. |
| | | MBq per patient max | Meredith R et al., |
| | | dosing | 2018. |
| ²²⁴Ra Radspherin (5) | 84.6/423* | Tolerable toxicity. | Example 5, model 1 |
| | | Assuming surface | |
| | | deposition. No | |
| | | product related | |
| | | toxicity observed. 7 | |
| | | MBq per patient max | |
| | | dosing. Following | |
| | | CRS. | |
| ²²⁴Ra Radspherin (5) | 39.6/198 | Tolerable toxicity. | Example 5, model 2, |
| | | Assuming volume | 200 ml liquid volume |
| | | distribution. No | |
| | | product related | |
| | | toxicity observed. 7 | |
| | | MBq/patient max | |
| | | dosing. Following | |
| | | CRS. | |

*Assuming a peritoneal surface area of 17500 cm².

Results: The Table 7 shows that Radspherin was given at much higher peritoneal equivalent doses compared with the other types of radiation therapy. It should be noted that external beam therapy and Radspherin groups consisted of patients treated concomitant after cytoreductive surgery.

### Example 10—Safety Aspects, the Avoidance of Radiation “Hot Spot” Toxicity

Background: When using radiolabelled micro-particles for therapy it is important to avoid negative effects from particle sediments which could cause so called “hot spots” of grains delivering a high local dose in a small spot. To reduce the risk of this, the specific activity of the micro-particles in terms of Bq per gram should be optimized.

Method and calculation results: A suspension of 7 MBq ²²⁴Ra bound to 0.7 gram of particles is typically used in the Radspherin therapy. If one assume a scenario where 10% of the particles generate a sediment that deposits locally on the surface of peritoneum, how much alpha-particle radiation would reach the peritoneum from the sediment?

It is assumed 2 g/cm3 as the particle density. Thus, the total injected volume of particle would be 0.7/2=0.35 cm³.

Assuming a 10% spheric grain of the infused particles, this has a weight of 0.035 cm³=35 mm³.

Using the equation: Volume, V=4/3 πr³, where V=volume and r=radius, the median particle radius is found to be 2.03 mm. The surface area of such a sphere would be, A=4 πr2=51.8 mm²

Assuming that the alpha radiation on the average has a maximum range, R, of 40 um=0.04 mm in the particle sphere the total volume of sediment material producing alpha radiation reaching the surface of the sphere would be Vr=A R=2.072 mm³. If one assumes that half of the alpha particles produced in the in the Vr is directed outwards to the surface and half is directed inwards to the sediment, the total activity reaching the surface would be (2.072 mm³/2)/35 mm³=0.03 that is 3%

If one assumes less than 50% of the sphere surface is in contact with the peritoneum, the percentage of alpha particles in the sediment sphere reaching the peritoneal tissue would be less than 1.5%.

In conclusion, by using sufficient materials as carrier for the alpha-emitting radioactivity, hot spot problems can be largely prevented.

### Example 11—Particle Characteristics

The median (numerical) diameter of the microparticles has been determined to about 3 μm. Assuming a 2 g/cm³ density and assuming a round sphere of 1.5 μm radius the volume of each sphere would be 14.1 μm³ according to the V=4/3 πr³ equation. Thus the number of particles in a dosing of 0.7 g of Radspherin would be 0.35 cm³/14.1 μm³=3.5×1011 μm³/14.1 μm³=2.48×10¹⁰.

The average number of ²²⁴Ra atoms in 0.7 gram of particle, N=A t_{1/2}/ln 2=7×10⁶×3.6×24×3600/ln 2=3.14×10¹² atoms (N—number of atoms; A—radioactivity; t_{1/2}—physical half-life of radionuclide).

The average number of ²²⁴Ra atoms per particle would then be: 3.14×10¹²/(2.48×10¹⁰)=126 atoms per microparticle.

Estimated surface area irradiated by one particle, the radiation zone: assuming a circle with radius of 25 um with the particle in center, the radiation zone surface area would be A=πr²=1963 μm². Assuming a peritoneal surface of 17500 cm²=17500×1×10⁸ μm²=1.75×10¹² μm².

The potential combined radiation zone area covered by the radiation from the microparticles would be: 2.48×10¹⁰×1963 μm2=4.86×10¹³ um².

There are about 28 times more particles then needed to cover the surfaces if assumed a perfect homogenous distribution on the peritoneal surfaces of the radiation zone. However, significant heterogeneous particle distribution may be expected as indicated by the gamma camera images and therefore the number of particles administered seems appropriate.

### Example 12—Parameter Comparisons for Dosing, Number of Atoms Per Particle and Antimetastatic Effect of Alpha-Emitter Labeled Microparticles

Methods: Patients received a fixed particle dosing with various amounts of ²²⁴Ra label. The number of atoms per particle, therapeutic effects and serious adverse events related to product is indicated for the different dosing groups.

TABLE 8
Therapy and toxicity data at 12 months after treatment of colorectal cancer
for Radspherin administered following cytoreductive surgery and HIPEC.

| | | Dosimetry* | | | Number of |
|---|---|---|---|---|---|
| ²²⁴Ra activity | | estimate of | | | Radspherin related^{#} |
| level per 0.7 | Estimated | peritoneal | Number of i.p. | | grade III or |
| gram of particles | average umber of | surface | metastases/number of | | higher adverse |
| (MBq) per | atoms per | equivalent | patients (% with i.p. | | events/number |
| patient | particle | dose (Sv) | metastases) | | of patients |
| 1 | 18 | 60 | 2/4 | (50%) | 0/4 |
| 2 | 36 | 120 | 1/3 | (33%) | 0/3 |
| 4 | 72 | 242 | 1/4 | (25%) | 0/4 |
| 7 | 126 | 423 | 0/12 | (0%) | 0/12 |

*From example 5, part 1, assuming surface deposition of ²²⁴Ra-labeled microparticles.
^{#}It should be noted that a small number of patients had grade III or higher adverse events related to the surgical procedure (Table 2, Example 4)

Results: The data presented in Table 8 shows that there is an indication of dose response in terms of frequency of i.p. metastases and at 7 MBq dosing no local metastases had been discovered up to the 12 month time point.

In conclusion: The data shows that a very high equivalent dose of ²²⁴Ra-Radspherin can be given resulting in promising suppression of local metastases development without indication of causing increased level of grade III or higher adverse events. Thus, an unexpectedly high tolerability to Radspherin alpha-radiation was demonstrated considering the equivalent dose level. The data also indicate that high equivalent doses are needed for maximizing the therapeutic gain.

We may speculate that the limiting dept dose of the short ranging alpha particles (<0.1 mm in tissue) and low chemical penetrability of the microparticles into the wound areas could be potential factors why it allows for the use of the unexpected, exceptionally high, equivalent doses, in Sv, with the described radionuclide-microparticle combination in this therapeutic setting. Also other aspects, like that some ²²⁰Rn release from the microparticles could cause a potential “dose smoothening” effect that could be advantageous from therapy and toxicity perspective, could be contributing to the clinical findings.

### Example 13—Safety Interim Results after Completion of the Dose-Limiting Toxicity (DLT) Period in a Dose-Escalation Study with Radspherin after Cytoreductive Surgery in Ovarian Cancer Patients

Methods: The outline of the study is described in Example 3. The dose escalation of Radspherin was performed at activity doses of 1-2-4-7-MBq.

Results: Fourteen patients were enrolled in the dose escalation cohort. All dose levels were well tolerated; no dose-limiting toxicity, no deaths, or discontinuations due to AEs were reported. This first analysis of safety in patients treated with Radspherin after secondary cytoreduction in patients with recurrent ovarian cancer found all dose levels to be well tolerated and DLT was not reached, no deaths occurred and only one Serious Adverse Event (SAE) related to Radspherin administration was reported. This grade 2 event was a procedural complication (leakage during administration) and was reported as SAE because being medically important. No complications have been identified during follow-up of the patient.

Conclusions: Radspherin was well tolerated at all dose levels and DLT was not reached in this dose-escalation study with intraperitoneal Radspherin treatment after cytoreductive surgery in ovarian cancer patients.

### Example 14—Eighteen-Month Safety and Efficacy after Intraperitoneal Treatment with Radspherin after Cytoreductive Surgery and Hyperthermic Intraperitoneal Chemotherapy for Colorectal Peritoneal Metastasis

Methods: The outline of the study is described in Example 1. Safety and efficacy signal of Radspherin injected intraperitoneally two days after CRS-HIPEC. After dose escalation (1-2-4-7 MBq), the 7 MBq dose was selected. Safety and efficacy assessments were performed every three months.

Results: Twenty-three patients were enrolled, one was excluded from the efficacy analysis, 12 patients received a dose of 7 MBq. Fourteen non-Radspherin related serious adverse events (SAEs) in eight patients have been reported. At 18-month, none of the 12 patients receiving 7 MBq had peritoneal recurrences, while 4 had non-peritoneal recurrences (33%). Across all doses, 9 out of 22 patients (41%) had recurred, whereof three patients recurred in the peritoneum.

Conclusions: Radspherin was well tolerated with no related SAEs reported. No patient receiving the highest dose tested of 7 MBq experienced peritoneal recurrence at 18 months in this study with peritoneal treatment of colorectal peritoneal metastasis after CRS-HIPEC.

### REFERENCES

Gu Q, Wang D, Cui C, Gao Y, Xia G, Cui X. Effects of radiation on wound healing. J Environ Pathol Toxicol Oncol. 1998; 17(2):117-23.
Koji Komori, Kenya Kimura, Takashi Kinoshita, Tsuyoshi Sano, Seiji Ito, Tetsuya Abe, Yoshiki Senda, Kazunari Misawa, Yuichi Ito, Norihisa Uemura, Ryosuke Kawai, and Yasuhiro Shimizu. Complications Associated With Postoperative Adjuvant Radiation Therapy for Advanced Rectal Cancer. Int Surg. 2014 March-April; 99(2): 100-105.
Li R G, Lindland K, Tonstad S K, Bønsdorff T B, Juzeniene A, Westrøm S, Larsen R H. Improved Formulation of ²²⁴Ra-Labeled Calcium Carbonate Microparticles by Surface Layer Encapsulation and Addition of EDTMP. Pharmaceutics. 2021 Apr. 29; 13(5):634.
Diaz C, Hayward C J et al., Ionizing Radiation Mediates Dose Dependent Effects Affecting the Healing Kinetics of Wounds Created on Acute and Late Irradiated Skin. Surgeries 2021, 2(1), 35-57. https://www.ncbi.nlm.nih.gov/pmc/articles/PMC7951225/
Haubner F, Ohmann E, Pohl F, Strutz J, and Gassner H G. Wound healing after radiation therapy: Review of the literature. Radiat Oncol. 2012; 7: 162. Published online 2012 Sep. 24. doi: 10.1186/1748-717X-7-162
Xueting Z, Yang H, LiYuan Z. The Detrimental Effect of Alpha Particle Radiation on Wound Healing and the Underlying Mechanism. Radiat Oncol Biol Phys. Poster Viewing Q&A Session|Volume 105, ISSUE 1, SUPPLEMENT, E661, Sep. 1, 2019
Vergote I B, Winderen M, De Vos L N, Tropé C G. Intraperitoneal radioactive phosphorus therapy in ovarian carcinoma. Analysis of 313 patients treated primarily or at second-look laparotomy. Cancer 1993 Apr. 1; 71(7):2250-60.
Fields E C, McGuire W P, Lin L, Temkin S M. Radiation Treatment in Women with Ovarian Cancer: Past, Present, and Future. Front. Oncol., 21 Aug. 2017
Sec. Radiation Oncology. https://doi.org/10.3389/fonc.2017.00177
Frøysnes I S, Larsen S G, Spasojevic M, Dueland S, Flatmark K. Complete cytoreductive surgery and hyperthermic intraperitoneal chemotherapy for colorectal peritoneal metastasis in Norway: Prognostic factors and oncologic outcome in a national patient cohort. J Surg Oncol. 2016 August; 114(2):222-7.
Arians N, Kieser M, Benner L, Rochet N et al. Arians N, Kieser M, Benner L, Rochet N, Schröder L, Katayama S, Herfarth K, Schubert K, Schneeweiss A, Sohn C, Lindel K, Debus J. Adjuvant intensity modulated whole-abdominal radiation therapy for high-risk patients with ovarian cancer FIGO stage III: final results of a prospective phase 2 study
Radiat Oncol. 2019 Oct. 21; 14(1):179.
Watson E E, Stabin M G, Davis J L, Eckerman K F. A model of the peritoneal cavity for use in internal dosimetry. J Nucl Med. 30:2002-2011, 1989.
Meredith R, Torgue J, Shen S, Fisher D R, Banaga E, Bunch P, Morgan D, Fan J, Straughn J M Jr. Dose escalation and dosimetry of first-in-human a radioimmunotherapy with ²¹²Pb-TCMC-trastuzumab. J Nucl Med. 2014 October; 55(10):1636-42.
Meredith R F, Torgue J J, Rozgaja T A, Banaga E P, Bunch P W, Alvarez R D, Straughn J M Jr, Dobelbower M C, Lowy A M. Safety and Outcome Measures of First-in-Human Intraperitoneal α Radioimmunotherapy With ²¹²Pb-TCMC-Trastuzumab. Am J Clin Oncol. 2018 July; 41(7):716-721.
Hallqvist A, Bergmark K, Bäck T, Andersson H, Dahm-Kähler P, Johansson M, Lindegren S, Jensen H, Jacobsson L, Hultborn R, Palm S, Albertsson P. Intraperitoneal α-Emitting Radioimmunotherapy with 211At in Relapsed Ovarian Cancer: Long-Term Follow-up with Individual Absorbed Dose Estimations. J Nucl Med. 2019 August; 60(8):1073-1079. doi: 10.2967/jnumed.118.220384. Epub 2019 Jan. 25. PMID: 30683761
Andersson H, Cederkrantz E, Bäck T, Divgi C, Elgqvist J, Himmelman J, Horvath G, Jacobsson L, Jensen H, Lindegren S, Palm S, Hultborn R. Intraperitoneal alpha-particle radioimmunotherapy of ovarian cancer patients: pharmacokinetics and dosimetry of (211)At-MX35 F(ab′)2—a phase I study. J Nucl Med. 2009 July; 50(7):1153-60.
Van der Pol J, Vöö S, Bucerius J, Mottaghy F M. Consequences of radiopharmaceutical extravasation and therapeutic interventions: a systematic review. Eur J Nucl Med Mol Imaging. 2017; 44(7): 1234-1243.
Dormand E L, Banwell P E, Goodacre T E E. Radiotherapy and wound healing. Int Wound J. 2005 June; 2(2): 112-127.

### ITEMS

1. A pharmaceutical composition comprising a therapeutically relevant amount of alpha emitting radionuclides.
2. The pharmaceutical composition according to item 1, wherein the alpha emitting radionuclides is dosage, and can be administered, with a radiation dose range of 6 Gy (30 Sv) to 2000 Gy (10 000 Sv), more specifically 10-200 Gy (50-1000 Sv).
3. The pharmaceutical composition according to any one or more of the previous items, wherein the radionuclide is selected from the group consisting of alpha-radionuclides suitable for therapy consisting of ²²⁴Ra, ²²⁵Ac, ²¹¹At, ²¹³Bi, ²¹²Bi, ²²³Ra, ¹⁴⁹Tb, ²²⁵Ra, ²³⁰U, ²⁵⁵Fm, and ²²⁷Th.
4. The pharmaceutical composition according to any one or more of the previous items, wherein the radionuclide is selected from the group consisting of alpha-emitting ²²⁴Ra with the progeny radionuclides ²²⁰Rn, ²¹⁶Po ²¹²Pb, ²¹²Bi, ²¹²Po and ²⁰⁸Tl.
5. The pharmaceutical composition according to any one or more of the previous items, wherein the radionuclide is a beta emitter with alpha-progenies suitable for therapy, which is ²¹²Pb with progeny radionuclides ²¹²Bi, ²¹²Po and ²⁰⁸Tl.
6. The pharmaceutical composition according to any one or more of the previous items, wherein the alpha emitting radionuclide is comprised in a particle, which can be biodegradable.
7. The pharmaceutical composition according to item 6, wherein the particle is comprising a degradable compound and the alpha emitting radionuclide.
8. The pharmaceutical composition according to items 6-7, wherein the particles furthermore comprise a phosphorus containing additive.
9. The pharmaceutical composition according to items 6-8, wherein the degradable compound is selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent.
10. The pharmaceutical composition according to items 6-9, wherein the degradable compound is CaCO₃, such as PEG modified CaCO₃, protein modified CaCO₃ including mAbs and Fabs, carbohydrate modified CaCO₃, lipid modified CaCO₃, vitamin modified CaCO₃, organic compound modified CaCO₃, polymer modified CaCO₃ and/or inorganic crystal modified CaCO₃.
11. The pharmaceutical composition according to items 8-10, wherein the phosphorus containing additive is a phosphate selected from the group consisting of orthophosphate, linear oligophosphates and polyphosphates, and cyclic polyphosphates.
12. The pharmaceutical composition according to items 8-11, wherein phosphorus containing additive is a polyphosphate selected from the group consisting of pyrophosphate, tripolyphosphate and triphosphono phosphate.
13. The pharmaceutical composition according to items 8-12, wherein the phosphorus containing additive is a cyclic polyphosphate which is sodium hexametaphosphate (SHMP).
14. The pharmaceutical composition according to items 8-13, wherein the phosphorus containing additive is a phosphonate.
15. The pharmaceutical composition according to items 8-14, wherein the phosphonate is a bisphosphonate.
16. The pharmaceutical composition according to items 8-15, wherein the bisphosphonate is selected from the group consisting of Etidronate, Clodronate, Tiludronate, Pamidronate, Neridronate, Olpadronate, Alendronate, Ibandronate, Risedronate, and Zoledronate.
17. The pharmaceutical composition according to items 8-16, wherein the phosphonate is a polyphosphonate.
18. The pharmaceutical composition according to item 17, wherein the polyphosphonate is selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid).
19. The pharmaceutical composition according to item 17, wherein the polyphosphonate is EDTMP-ethylenediamine tetra(methylene phosphonic acid).
20. The pharmaceutical composition according to item 6, wherein the particle is comprising one or more compounds selected from the group consisting of a polymer, alginate and crystalline salt (including salt of sulfate), silica, phosphate, carbonate, gelatin, polystyrene, lactate, and barium sulfate.
21. The pharmaceutical composition according to any one or more of the previous items, wherein the size of the particle is from 1 nm to 500 μm.
22. The pharmaceutical composition according to any one or more of the previous items, wherein the pharmaceutical composition is comprising one or more particles according to any one of claims 6-20 and a diluent, carrier, surfactant, and/or excipient.
23. The pharmaceutical composition according to any one or more of the previous items, prepared with an amount of radionuclide that is 1 kBq to 10 GBq per dosing or with an amount of radionuclide that is 50 MBq to 100 GBq suitable for multidose industrial scale production.
24. The pharmaceutical composition according to any one or more of the previous items, wherein the composition is a particle suspension comprising monodisperse or polydisperse particles as defined in items 6-22.
25. The pharmaceutical composition according to any one or more of the previous items for use in the treatment of cancer.
26. The pharmaceutical composition for use according to any one or more of the previous items, wherein the cancer is selected from the group consisting of intraperitoneal cancers, intracranial cancers, pleural cancers, bladder cancers, cardiac cancers, cancers in the subarachnoid cavity, and non-cavitary targets such as melanoma, non-small-cell-lung cancer, and metastasis.
27. The pharmaceutical composition according to any one or more of the previous items, wherein the individual has received cytoreductive surgery of one or more peritoneal tumor(s) prior to the administration of the pharmaceutical composition.
28. The pharmaceutical composition for use according to any one or more of the previous items, wherein the cytoreductive surgery of one or more peritoneal tumor(s) is performed preoperatively, e.g., the same day, one day prior to the administration of the pharmaceutical composition comprising a therapeutically relevant amount of alpha emitting radionuclides, such as at least two days, such as at least three days, such as at least four days, such as at least five days, such as at least six days, such as at least seven days, such as at least eight days, such as at least nine days, such as at least ten days, such as at least eleven days, such as at least twelve days, such as at least thirteen days, such as at least fourteen days.
29. The pharmaceutical composition for use according to any one or more of the previous items, wherein the pharmaceutical composition is used in combination with other cancer therapies, such as chemotherapy like taxanes (e.g. paclitaxel, docetaxel), platins (e.g. carboplatin, cisplatin), doxorubicin, mitomycin), DNA repair inhibitors such as PARP inhibitors (e.g. Olaparib, Rucaparib, Niraparib, Talazoparib, Veliparib, Pamiparib, CEP 9722, E7016, and 3-Aminobenzamide), and radioimmunotherapies.
30. The pharmaceutical composition for use according to any one or more of the previous items, wherein the pharmaceutical composition is a medical device or is comprised in a medical device.
31. The pharmaceutical composition according to any one or more of the previous items, wherein the concentrations of phosphonates and or phosphate compounds are 1 microgram to 1000 milligram per ml, such as 0.1 mg to 10 mg per ml of final solution, or 1 microgram to 1000 milligram per gram particles in the final solution.
32. The pharmaceutical composition according to any one or more of the previous items, wherein 0.1-10 gram of particles is used per dosing, such as 0.5-5 gram of particles per dosing.
33. The pharmaceutical composition according to any one or more of the previous items, wherein there is 2 MBq to 50 MBq of ²²⁴Ra per gram of microparticles.
34. The pharmaceutical composition according to any one or more of the previous items, wherein the average particle size is 0.1-30 um, such as 1-10 um, and which is radiolabelled with 1-2000 atoms of alpha emitter per particle, such as 50-500 atoms per micro-particle.
35. The pharmaceutical composition for use according to any one or more of the previous items, wherein the individual has received Hyperthermic intraperitoneal chemotherapy (HIPEC).
36. The pharmaceutical composition for use according to any one or more of the previous items, wherein the pharmaceutical composition is administered to an individual in needed thereof.
37. The pharmaceutical composition for use according to any one or more of the previous items, wherein the pharmaceutical composition is infused into the peritonel cavity of a patient recently treated by cytoreductive surgery with or without HIPEC.
38. The pharmaceutical composition for use according to any one or more of the previous items, wherein the tumor(s) is one or more peritoneal tumor(s).
39. A particle of numerical diameter size 0.1-30 um, such as 1-10 um, radiolabelled with 1-2000 atoms of alpha emitter per particle, such as 50-500 atoms per micro-particle.
40. A particle according to item 38 radiolabelled with an alpha emitter, such as ²²⁴Ra, and administered as a suspension using a radioactivity dosage of 1-50 MBq, such as 4-20 MBq, such as 5-10 MBq per patient.
41. A suspension of alpha emitter-labeled particles, such as crystalline particles, according to any of the previous items, containing 50-3000 mg such as 500-1500 mg for intraperitoneal infusion and with an activity of 1 MBq-500 MBq, such as 4-12 MBq and dispersed in 1-2000 ml such as 10-500 ml such as 100-300 ml.

1. A pharmaceutical composition comprising a therapeutically effective amount of alpha emitting radionuclides, wherein the alpha emitting radionuclides are at a dosage that can be administered with a radiation dose range of 6 Gy (30 Sv) to 2000 Gy (10 000 Sv).

2-23. (canceled)

24. The pharmaceutical composition according to claim 1, wherein the radionuclides are selected from the group consisting of ²²⁴Ra, ²²⁵Ac, ²¹¹At, ²¹³Bi, ²¹²Bi, ²²³Ra, ¹⁴⁹Tb, ²²⁵Ra, ²³⁰U, ²⁵⁵Fm, and ²²⁷Th.

25. The pharmaceutical composition according to claim 1, wherein the radionuclides comprise ²²⁴Ra with the progeny radionuclides ²²⁰Rn, ²¹⁶Po, ²¹²Pb, ²¹²Bi, ²¹²Po and ²⁰⁸Tl.

26. The pharmaceutical composition according to claim 1, wherein the radionuclide is ²¹²Pb with progeny radionuclides ²¹²Bi, ²¹²Po and ²⁰⁸Tl.

27. The pharmaceutical composition according to claim 1, containing 50-3000 mg of alpha emitting radionuclides formulated for intraperitoneal infusion and with an activity of 1 MBq-500 MBq and dispersed in 1-2000 ml.

28. The pharmaceutical composition according to claim 1, further comprising a phosphorus containing additive.

29. The pharmaceutical composition according to claim 1, further comprising a degradable compound selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent.

30. The pharmaceutical composition according to claim 1, comprising ²²⁴Ra, EDTMP, and CaCO₃.

31. A method for treating cancer comprising administering a therapeutically effective amount of the pharmaceutical composition of claim 1 to a subject in need thereof.

32. The method of claim 31, wherein the cancer is selected from the group consisting of intraperitoneal cancers, intracranial cancers, pleural cancers, bladder cancers, cardiac cancers, cancers in the subarachnoid cavity, and non-cavitary targets such as melanoma, non-small-cell-lung cancer, and metastasis.

33. The method of claim 31, wherein the subject has received cytoreductive surgery of one or more peritoneal tumor(s) prior to the administration of the pharmaceutical composition.

34. The method of claim 31, wherein 0.1-10 gram of alpha emitting radionuclides are administered per dosing.

35. The method of claim 31, wherein there is 2 MBq to 50 MBq of ²²⁴Ra per gram of microparticles.

36. The pharmaceutical composition according to claim 1, wherein the average particle size is 0.1-30 micron and radiolabelled with 1-2000 atoms of alpha emitter per particle.

37. A particle of numerical diameter size 0.1-30 micron radiolabelled with 1-2000 atoms of alpha emitter per particle.
